(19) United States
(12) Patent Application Publication
(10) Pub. No.: US 2005/0112584 A1
(43) Pub. Date: May 26, 2005

(54) Methods for evaluating tissue pair combinations for use in nucleic acid array technology

(76) Inventors: Francisco J. Cifuentes, San Francisco, CA (US); Meha Kapadia, San Francisco, CA (US); Eric M. Leproust, San Jose, CA (US); Stephanie B. Fulmer-Smentek, Sunnyvale, CA (US); Bill J. Peck, Mountain View, CA (US)

Correspondence Address: AGILENT TECHNOLOGIES, INC.;Legal Department, DL429, Intellectual Property Administration, P.O. Box 7599, Loveland, CO, 80537-0599, US

(21) Appl. No.: 10/718,342

(22) Filed: Nov. 20, 2003

(51) Int. Cl. C12Q 1/68; G06F 19/00; G01N 33/48; G01N 33/50

(52) U.S. Cl. 435/6; 702/20

(57) ABSTRACT

Methods are disclosed for selecting a combination of nucleic acid sample pairs for evaluating the ability of an oligonucleotide probe to measure differential expression of genes. Differential gene expression experiments are conducted using (i) nucleic acid sample pairs and (ii) nucleic acid probes immobilized on a substrate, the probes representing a set of genes. The number of genes in the set is a portion of an expected number of genes in a sample. A nucleic acid sample pair combination is selected based on the members of the combination having a maximized number of genes from the set of genes that exhibit differential expression and a minimized number of the genes that do not exhibit differential expression.

### BACKGROUND OF THE INVENTION

Molecular methods using DNA probes, nucleic acid hybridizations and in vitro amplification techniques are promising methods offering advantages to conventional methods used for patient diagnoses, biomedical research or basic biology research. Recent advances in such methods often include the introduction of parallelism, i.e., performing many experiments with the same effort previously used to perform a single experiment. However, the introduction of parallelism often forces changes in the methods used to design such experiments.

Nucleic acid hybridization has been employed for investigating the identity and establishing the presence of nucleic acids. Hybridization is based on complementary base pairing. When complementary single stranded nucleic acids are incubated together, the complementary base sequences pair to form double stranded hybrid molecules. The ability of single stranded deoxyribonucleic acid (ssDNA) or ribonucleic acid (RNA) to form a hydrogen bonded structure with a complementary nucleic acid sequence has been employed as an analytical tool in molecular biology research. The availability of radioactively, chemically and fluorescently labeled nucleoside triphosphates of high specific activity have made it possible to identify, isolate, and characterize various nucleic acid sequences of biological interest. Nucleic acid hybridization has great potential in diagnosing or characterizing diseased or altered tissue function associated with unique nucleic acid sequences or gene expression states. Unique nucleic acid sequences may result from genetic or environmental change in DNA by insertions, deletions, point mutations, or by acquiring foreign DNA or RNA by means of infection by bacteria, molds, fungi, and viruses. Altered gene expression states may arise from neoplastic transformation, viral infection, environmental insult or drug treatment. It is desirable to perform such experiments in parallel; earlier methods for introducing modest parallelism include Southern blots, Northern blots and slot blots.

Such blot techniques are examples of methods for detecting nucleic acids that employ nucleic acid probes that have sequences complementary to sequences in the target nucleic acid. A nucleic acid probe may be, or may be capable of being, labeled with a reporter group or may be, or may be capable of becoming, bound to a support. Detection of signal depends upon the nature of the label or reporter group. Usually, the probe is comprised of natural nucleotides such as ribonucleotides and deoxyribonucleotides and their derivatives although unnatural nucleotide mimetics such as peptide nucleic acids and oligomeric nucleoside phosphonates are also used.

Commonly, binding of the probes to the target is detected by means of a label incorporated into the probe. Alternatively, the probe may be unlabeled and the target nucleic acid labeled. Binding can be detected by separating the bound probe or target from the free probe or target and detecting the label. In one approach, a sandwich is formed comprised of one probe, which may be labeled, the target and a probe that is or can become bound to a surface. Alternatively, binding can be detected by a change in the signal-producing properties of the label upon binding, such as a change in the emission efficiency of a fluorescent or chemiluminescent label. This permits detection to be carried out without a separation step. Finally, binding can be detected by labeling the target, allowing the target to hybridize to a surface-bound probe, washing away the unbound target and detecting the labeled target that remains.

Direct detection of labeled target hybridized to surface-bound probes is particularly advantageous if the surface contains a mosaic of different probes that are individually localized to discrete, known areas of the surface. Arrays of binding agents or probes, such as polypeptide and nucleic acids, have become an increasingly important tool in the biotechnology industry and related fields. These binding agent arrays find use in a variety of different fields, e.g., genomics (in sequencing by hybridization, SNP detection, differential gene expression analysis, high throughput analyses of genotype, identification of novel genes, gene mapping, finger printing, etc.) and proteomics. Ordered arrays containing a large number of oligonucleotide probes on a solid support recognize uniquely complementary nucleic acids by hybridization, and arrays can be designed to define specific target sequences, analyze gene expression patterns or identify specific allelic variations.

In using such arrays, the surface bound probes are contacted with molecules or analytes of interest, i.e., targets, in a sample. Targets in the sample bind to the complementary probes on the substrate to form a binding complex. The pattern of binding of the targets to the probe features or spots on the substrate produces a pattern on the surface of the substrate and provides desired information about the sample. In most instances, as mentioned above, the targets are labeled with a detectable label or reporter such as a fluorescent label, chemiluminescent label or radioactive label. The resultant binding interaction or complexes of binding pairs are then detected and read or interrogated, for example by optical means, although other methods may also be used depending on the detectable label employed. For example, laser light may be used to excite fluorescent labels bound to a target, generating a signal only in those spots on the substrate that have a target, and thus a fluorescent label, bound to a probe molecule. This pattern may then be digitally scanned for computer analysis.

Generally, in discovering or designing probes to be used in an array, a nucleic acid sequence is selected based on the particular gene of interest, where the nucleic acid sequence may be as great as about 60 or more nucleotides in length or as small as about 25 nucleotides in length or less. From the nucleic acid sequence, probes are synthesized according to various nucleic acid sequence regions, i.e., subsequences, of the nucleic acid sequence and are associated with a substrate to produce a nucleic acid array. As described above, a detectably labeled sample is contacted with the array, where targets in the sample bind to complimentary probe sequences of the array.

An important step in designing arrays is the selection of a specific probe or mixture of probes that may be used in the array. Such probes should maximize the chances of binding with a specific target in a sample and at the same time should minimize the time and expense involved in probe discovery and design.

For example, conventional probe design may be performed experimentally or computationally, where in many instances it is performed computationally. Accordingly, probe design usually involves taking subsequences of a nucleic acid and filtering them based on certain computationally determined values such as melting temperature, self structure, homology, etc., to attempt to predict which subsequences will generate probes that will provide good signal and/or will not cross-hybridize. The subsequences that remain after the filtering process are selected to generate probes to be used in nucleic acid arrays.

One difficulty in the design of oligonucleotide arrays is that oligonucleotides targeted to different regions of the same gene can show large differences in hybridization efficiency, presumably due to the interplay between the secondary structures of the oligonucleotides and their targets and the stability of the final probe/target hybridization product.

In one approach to validating probes, candidate probe sequences are evaluated for their performance under a plurality of different experimental sets, specifically a plurality of differential gene expression experiments to obtain a collection of empirically obtained performance data values for each of the candidate nucleic acid probe sequences for each of the plurality of different experimental conditions. Such an approach utilizes tissue pair combinations.

There is continued interest in the development of methods for validating nucleic acid probes with regards to differential gene expression including methods for selecting optimal tissue pair combinations.

### SUMMARY OF THE INVENTION

One embodiment of the present invention is a method for selecting a combination of nucleic acid sample pairs for evaluating the ability of an oligonucleotide probe to measure differential expression of genes. Differential gene expression experiments are conducted using (i) nucleic acid sample pairs and (ii) nucleic acid probes immobilized on a substrate, the probes representing a set of genes. The number of genes in the set is a portion of an expected number of genes in a sample. A nucleic acid sample pair combination is selected based on the members of the combination having the most number of genes from the set of genes that exhibit differential expression and the least number of the genes that do not exhibit differential expression.

Another embodiment of the present invention is a method for selecting a combination of nucleic acid sample pairs for evaluating the ability of an oligonucleotide probe to measure differential expression of genes. Each nucleic acid sample pair is contacted with a plurality of probes for each of a predetermined number of genes to determine whether the genes exhibit differential expression. A determination is made for each gene and each of the nucleic acid sample pairs whether the gene exhibits differential expression based on one or more criteria. For a gene that exhibits differential expression for a nucleic acid sample pair, a “yes” value is assigned, and for each gene that does not exhibit differential expression for a nucleic acid sample pair, a “no” value is assigned. The above steps are repeated for the number of nucleic acid sample pairs to be evaluated. The data is tabulated for each combination of nucleic acid sample pairs to be evaluated. A combination of nucleic acid sample pairs is selected having a score based on a maximized number of “yes's” and a minimized number of “no's.”

Another embodiment of the present invention relates to the method described above where the results of the contacting are analyzed utilizing an analysis system comprising a computer.

Another embodiment of the present invention is a method for selecting a combination of nucleic acid sample pairs for evaluating the ability of an oligonucleotide probe to measure differential expression of genes. Each nucleic acid sample pair from a plurality of nucleic acid sample pairs is contacted with a plurality of probes for each of a predetermined number of genes to determine whether the genes exhibit differential expression. A determination is made for each gene and each of the nucleic acid sample pairs whether the gene exhibits or does not exhibit differential expression based on one or more parameters. The results of the contacting and the determining are analyzed utilizing an analysis system comprising a computer. The computer comprises (i) means for assigning, for each gene and to each of the nucleic acid sample pairs, a value of 1 if the gene exhibits differential expression or assigning a value of 0 if the gene does not exhibit differential expression, (ii) means for tabulating data from step (i) to determine the total number scores for each combination of the nucleic acid sample pairs to be evaluated, wherein the maximum number of 1's for each nucleic acid sample pair per gene is the number “n” of nucleic acid sample pairs of the combination, and (iii) means for selecting a combination of nucleic acid sample pairs based on a score representing a maximized number of n's and a minimized number of 0's.

Another embodiment of the present invention is a computer-based method for selecting a combination of nucleic acid sample pairs for evaluating the ability of an oligonucleotide probe to measure differential expression of genes. Each nucleic acid sample pair from a plurality of nucleic acid sample pairs is contacted with a plurality of probes for each of a predetermined number of genes to determine whether the genes exhibit differential expression. A determination is made under computer control for each gene and each of the nucleic acid sample pairs whether the gene exhibits or does not exhibit differential expression based on one or more parameters. Under computer control, a “yes” value is assigned for a gene that exhibits differential expression for a nucleic acid sample pair and a “no” value is assigned for each gene that does not exhibit differential expression for a nucleic acid sample pair. Under computer control, data from above is tabulated for each combination of the nucleic acid sample pairs to be evaluated. A combination of nucleic acid sample pairs is selected under computer control where the combination of nucleic acid sample pairs has a score based on a maximized number of “yes's” and a minimized number of “no's.”

Another embodiment of the present invention is a method of identifying a sequence of a nucleic acid that is suitable for use as a substrate surface immobilized probe for a target nucleic acid. A plurality of candidate probe sequences for the target nucleic acid is identified based on at least one selection criterion. Each of the candidate probe sequences is empirically evaluated under a plurality of different experimental sets to obtain a collection of empirical data values for each of the candidate nucleic acid probe sequences for each of the plurality of different experimental sets wherein the empirical evaluation employs a nucleic acid sample pair selected by a method as described above. The candidate probe sequences are clustered into one or more groups of candidate probe sequences based on each candidate probe sequence's collection of empirical data values, wherein each of the one or more groups exhibits substantially the same performance across the plurality of experimental sets. One or more groups are selected based on at least one criterion. A candidate probe sequence is chosen from the selected group as the sequence of the nucleic acid that is suitable for use as a substrate immobilized probe for the target nucleic acid.

Another embodiment of the present invention is a method of producing an array of nucleic acids on the surface of a substrate. Nucleic acid probes are identified by a method as described above. The nucleic acid probes identified above are synthesized or deposited in the form of an array on the surface of a substrate.

Another embodiment of the present invention is a method of detecting the presence of a nucleic acid analyte in a sample. A nucleic acid array produced as described above is contacted with a sample. The presence of binding complexes on the surface of the array is detected, the presence thereof indicating the presence of the nucleic acid analyte in the sample.

Another embodiment of the present invention is a kit for selecting a combination of nucleic acid sample pairs for evaluating the ability of an oligonucleotide probe to measure differential expression of genes. The kit comprises (a) an algorithm for use in conducting differential gene expression experiments using (i) nucleic acid sample pairs and (ii) nucleic acid probes immobilized on a substrate where the probes represent a set of genes. The number of genes in the set is a portion of an expected number of genes in a sample. The algorithm is present on a computer readable medium. The kit also includes instructions for using the algorithm to select a nucleic acid sample pair combination based on the members of the combination having a maximized number of genes from the set of genes that exhibit differential expression and a minimized number of the genes that do not exhibit differential expression.

### BRIEF DESCRIPTION OF THE DRAWINGS

FIG. 1 is a perspective view of a substrate bearing multiple arrays, as may be produced by a method and apparatus of the present invention.

FIG. 2 is an enlarged view of a portion of FIG. 1 showing some of the identifiable individual regions (or “features”) of a single array of FIG. 1.

FIG. 3 is an enlarged cross-section of a portion of FIG. 2.

### DESCRIPTION OF SPECIFIC EMBODIMENTS OF THE INVENTION

As indicated above, the present invention may be employed for selecting an optimal nucleic acid sample pair combination for validating probes for their use in analyses involving differential gene expression. While the present invention is described herein with reference to a particular probe validation method, this is by way of illustration and not limitation. The present invention may be applied to instances where it is desired to optimize evaluation methods involving nucleic acid sample pair combinations. Such instances include, for example, comparison of microarray platforms, analysis of microarray performance, and the like.

Furthermore, the subject invention provides methods of identifying or designing probes for use in array structures, where the probes are chemical probes, e.g., biopolymer probes, such as nucleic acid probes. While the following description is provided in terms of nucleic acid probe design protocols for ease and clarity of description, the scope of the invention is not so limited and extends to the identification or design of suitable probes for use in any type of array structure.

In the present method differential expression experiments are conducted using (i) nucleic acid sample pair combinations representing a predetermined number of nucleic acid sample pairs and (ii) nucleic acid probes immobilized on a substrate. The probes represent a set of genes where the number of genes in the set is a portion of an expected number of genes in a sample. A nucleic acid sample pair combination is selected in relation to the members of the combination having the most number of genes from the set of genes that exhibit differential expression and the least number of the genes that do not exhibit differential expression.

Terminology

Before proceeding further with a description of the specific embodiments of the present invention, a number of terms will be discussed. In the present application, unless a contrary intention appears, the following terms refer to the indicated characteristics.

The term “polymer” means any compound that is made up of two or more monomeric units covalently bonded to each other, where the monomeric units may be the same or different, such that the polymer may be a homopolymer or a heteropolymer.

The term “biopolymer” refers to a polymer of biological significance. Biopolymers are typically found in biological systems and particularly include polysaccharides (such as carbohydrates), and peptides (which term is used to include polypeptides and proteins) and polynucleotides as well as their analogs such as those compounds composed of or containing amino acid analogs or non-amino acid groups, or nucleotide analogs or non-nucleotide groups. This includes polynucleotides in which the conventional backbone has been replaced with a non-naturally occurring or synthetic backbone, and nucleic acids (or synthetic or naturally occurring analogs) in which one or more of the conventional bases has been replaced with a group (natural or synthetic) capable of participating in Watson-Crick type hydrogen bonding interactions. Polynucleotides include single or multiple stranded configurations, where one or more of the strands may or may not be completely aligned with another. For example, a “biopolymer” includes DNA (including cDNA), RNA, oligonucleotides, and PNA and other polynucleotides as described in U.S. Pat. No. 5,948,902 and references cited therein (all of which are incorporated herein by reference), regardless of the source.

The term “nucleic acid” as used herein means a polymer composed of nucleotides, e.g., deoxyribonucleotides or ribonucleotides, or compounds produced synthetically (e.g. PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “oligonucleotide” refers to a nucleotide multimer of about 10 to 100 nucleotides in length and up to 200 nucleotides in length.

The term “polynucleotide” as used herein refers to a nucleotide multimer having any number of nucleotides. The polynucleotides include nucleic acids, and fragments thereof, from any source in purified or unpurified form including DNA (dsDNA and ssDNA) and RNA, including tRNA, mRNA, rRNA, mitochondrial DNA and RNA, chloroplast DNA and RNA, DNA/RNA hybrids, or mixtures thereof, genes, chromosomes, plasmids, cosmids, the genomes of biological material such as microorganisms, e.g., bacteria, yeasts, phage, chromosomes, viruses, viroids, molds, fungi, plants, animals, humans, and the like.

The term “biomonomer” references a single unit, which can be linked with the same or other biomonomers to form a biopolymer (for example, a single amino acid or nucleotide with two linking groups one or both of which may have removable protecting groups). The terms “biomonomer fluid” and “biopolymer fluid” reference a liquid containing either a biomonomer or biopolymer, respectively (typically in solution).

The term “monomer” as used herein refers to a chemical entity that can be covalently linked to one or more other such entities to form a polymer. Examples of “monomers” include nucleotides, amino acids, saccharides, peptides, other reactive organic molecules and the like. In general, the monomers used in conjunction with the present invention have first and second sites (e.g., C-termini and N-termini(for proteins), or 5′ and 3′ sites(for oligomers, RNA's, cDNA's, and DNA's)) suitable for binding to other like monomers by means of standard chemical reactions (e.g., condensation, nucleophilic displacement of a leaving group, or the like), and a diverse element which distinguishes a particular monomer from a different monomer of the same type (e.g., an amino acid side chain, a nucleotide base, etc.). In the art synthesis of biomolecules of this type utilize an initial substrate-bound monomer that is generally used as a building-block in a multi-step synthesis procedure to form a complete ligand, such as in the synthesis of oligonucleotides, oligopeptides, and the like.

The term “oligomer” is used herein to indicate a chemical entity that contains a plurality of monomers. As used herein, the terms “oligomer” and “polymer” may be used interchangeably. Examples of oligomers and polymers include polydeoxyribonucleotides (DNA), polyribonucleotides (RNA), other polynucleotides, which are C-glycosides of a purine or pyrimidine base, polypeptides (proteins), polysaccharides (starches, or polysugars), and other chemical entities that contain repeating units of like chemical structure.

The term “sample” as used herein relates to a material or mixture of materials, typically, although not necessarily, in fluid form, containing one or more targets, i.e., components or analytes of interest.

The phrase “nucleic acid sample pair” means a pair of samples where each member of the sample pair comprises a plurality of nucleic acid targets. The targets may be gene products or any synthetic RNA and so forth. The nucleic acid sample may be from any source such as human or other animal and so forth. The nucleic acid sample may be tissue from any organ or mix of organs such as kidney, liver, spleen, brain and so forth or cell line from any healthy or defective organ, such as HELA, K562 and the like.

The terms “nucleoside” and “nucleotide” refer to a sub-unit of a nucleic acid and has a phosphate group, a 5-carbon sugar and a nitrogen containing base, as well as functional analogs (whether synthetic or naturally occurring) of such sub-units which in the polymer form (as a polynucleotide) can hybridize with naturally occurring polynucleotides in a sequence specific manner analogous to that of two naturally occurring polynucleotides. The terms “nucleoside” and “nucleotide” are intended to include those moieties, which contain not only the known purine and pyrimidine bases, but also other heterocyclic bases that have been modified. Such modifications include methylated purines or pyrimidines, acylated purines or pyrimidines, alkylated riboses or other heterocycles. In addition, the terms “nucleoside” and “nucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like.

The terms “may” “optional” or “optionally” used herein interchangeably means that the subsequently described circumstance may or may not occur, so that the description includes instances where the circumstance occurs and instances where it does not.

The terms “probe”, “probe sequence”, “target probe” or “ligand” as used herein refer to a moiety made of an oligonucleotide or polynucleotide, as defined above, which contains a nucleic acid sequence complementary to a nucleic acid sequence present in a sample of interest such that the probe will specifically hybridize to the nucleic acid sequence present in the sample under appropriate conditions. In some embodiments, the nucleic acid probes are typically associated with a support or substrate to provide an array of nucleic acid probes to be used in an array assay. The term “probe” or its equivalents as used herein refer to a compound that is “pre-synthesized” or obtained commercially, and then attached to the substrate or synthesized on the substrate, i.e., synthesized in situ on the substrate. The nucleic acid probes may be produced, generated or synthesized according to probe sequences identified as suitable according to the subject invention that may or may not have been further tested or characterized.

The terms “reporter”, “label” “detectable reporter” and “detectable label” are used herein to refer to a molecule capable of detection, including, but not limited to, radioactive isotopes, fluorescers, chemiluminescers, enzymes, enzyme substrates, enzyme cofactors, enzyme inhibitors, dyes, metal ions, metal sols, other suitable detectable markers such as biotin or haptens and the like. The term “fluorescer” refers to a substance or portion thereof which is capable of exhibiting fluorescence in the detectable range. The term “cofactor” is used broadly herein to include any molecular moiety that participates in an enzymatic reaction. Particular example of labels which may be used under the invention include, but are not limited to, fluorescein, 5(6)-carboxyfluorescein, Cyanine 3 (Cy3), Cyanine 5 (Cy5), rhodamine, dansyl, umbelliferone, Texas red, luminal, NADPH, horseradish peroxidase and α,β-galactosidase.

An “array,” includes any two-dimensional or substantially two-dimensional (as well as a three-dimensional) arrangement of addressable regions bearing a particular chemical moiety or moieties (e.g., biopolymers such as polynucleotide or oligonucleotide sequences (nucleic acids), polypeptides (e.g., proteins), carbohydrates, lipids, etc.) associated with that region. In the broadest sense, the preferred arrays are arrays of polymeric binding agents, where the polymeric binding agents may be any of: polypeptides, proteins, nucleic acids, polysaccharides, synthetic mimetics of such biopolymeric binding agents, etc. In many embodiments of interest, the arrays are arrays of nucleic acids, including oligonucleotides, polynucleotides, cDNA's, mRNA's, synthetic mimetics thereof, and the like. Where the arrays are arrays of nucleic acids, the nucleic acids may be covalently attached to the arrays at any point along the nucleic acid chain, but are generally attached at one of their termini (e.g. the 3′ or 5′ terminus). Sometimes, the arrays are arrays of polypeptides, e.g., proteins or fragments thereof.

Any given substrate may carry one, two, four or more or more arrays disposed on a front surface of the substrate. Depending upon the use, any or all of the arrays may be the same or different from one another and each may contain multiple spots or features. A typical array may contain more than ten, more than one hundred, more than one thousand, more ten thousand features, or even more than one hundred thousand features, in an area of less than 20 cm² or even less than 10 cm². For example, features may have widths (that is, diameter, for a round spot) in the range from a 10 μm to 1.0 cm. In other embodiments each feature may have a width in the range of 1.0 μm to 1.0 mm, usually 5.0 μm to 500 μm, and more usually 10 μm to 200 μm. Non-round features may have area ranges equivalent to that of circular features with the foregoing width (diameter) ranges. At least some, or all, of the features are of different compositions (for example, when any repeats of each feature composition are excluded the remaining features may account for at least 5%, 10%, or 20% of the total number of features). Interfeature areas will typically (but not essentially) be present which do not carry any polynucleotide (or other biopolymer or chemical moiety of a type of which the features are composed). Such interfeature areas typically will be present where the arrays are formed by processes involving drop deposition of reagents but may not be present when, for example, photolithographic array fabrication processes are used. It will be appreciated though, that the interfeature areas, when present, could be of various sizes and configurations.

Each array may cover an area of less than 100 cm², or even less than 50 cm², 10 cm² or 1 cm². In many embodiments, the substrate carrying the one or more arrays will be shaped generally as a rectangular solid (although other shapes are possible), having a length of more than 4 mm and less than 1 m, usually more than 4 mm and less than 600 mm, more usually less than 400 mm; a width of more than 4 mm and less than 1 m, usually less than 500 mm and more usually less than 400 mm; and a thickness of more than 0.01 mm and less than 5.0 mm, usually more than 0.1 mm and less than 2 mm and more usually more than 0.2 and less than 1 mm. With arrays that are read by detecting fluorescence, the substrate may be of a material that emits low fluorescence upon illumination with the excitation light. Additionally in this situation, the substrate may be relatively transparent to reduce the absorption of the incident illuminating laser light and subsequent heating if the focused laser beam travels too slowly over a region. For example, substrate 10 may transmit at least 20%, or 50% (or even at least 70%, 90%, or 95%), of the illuminating light incident on the front as may be measured across the entire integrated spectrum of such illuminating light or alternatively at 532 nm or 633 nm.

As mentioned above, an individual support may contain a single array or multiple arrays. Features of the array may be arranged in rectilinear rows and columns. This is particularly attractive for single arrays on a support. When multiple arrays are present, such arrays can be arranged, for example, in a sequence of curvilinear rows across the substrate surface (for instance, a sequence of concentric circles or semi-circles of spots), and the like. Similarly, the pattern of features may be varied from the rectilinear rows and columns of spots to include, for example, a sequence of curvilinear rows across the support surface (for example, a sequence of concentric circles or semi-circles of spots), and the like. The configuration of the arrays and their features may be selected according to manufacturing, handling, and use considerations.

Arrays can be fabricated using drop deposition from pulsejets of either polynucleotide precursor units (such as monomers) in the case of in situ fabrication, or the previously obtained polynucleotide. Such methods are described in detail in, for example, the previously cited references including U.S. Pat. Nos. 6,242,266, 6,232,072, 6,180,351, 6,171,797, 6,323,043, U.S. patent application Ser. No. 09/302,898 filed Apr. 30, 1999 by Caren, et al., and the references cited therein. These references are incorporated herein by reference. Other drop deposition methods can be used for fabrication, as previously described herein. In addition, instead of drop deposition methods, photolithographic array fabrication methods may be used. Interfeature areas need not be present particularly when the arrays are made by photolithographic methods as described in those patents.

An array is “addressable” when it has multiple regions of different moieties (e.g., different polynucleotide sequences) such that a region (i.e., a “feature” or “spot” of the array) at a particular predetermined location (i.e., an “address”) on the array will detect a particular target or class of targets (although a feature may incidentally detect non-targets of that feature). Array features are typically, but need not be, separated by intervening spaces. In the case of an array, the “target” will be referenced as a moiety in a mobile phase (typically fluid), to be detected by probes (“target probes”) which are bound to the substrate at the various regions. However, either of the “target” or “target probe” may be the one which is to be evaluated by the other (thus, either one could be an unknown mixture of polynucleotides to be evaluated by binding with the other). A “scan region” refers to a contiguous (preferably, rectangular) area in which the array spots or features of interest, as defined above, are found. The scan region is that portion of the total area illuminated from which the resulting fluorescence is detected and recorded. For the purposes of this invention, the scan region includes the entire area of the slide scanned in each pass of the lens, between the first feature of interest, and the last feature of interest, even if there exist intervening areas that lack features of interest. An “array layout” refers to one or more characteristics of the features, such as feature positioning on the substrate, one or more feature dimensions, and an indication of a moiety at a given location.

The terms “hybridization (hybridizing)” and “binding” in the context of nucleotide sequences are used interchangeably herein. The ability of two nucleotide sequences to hybridize with each other is based on the degree of complementarity of the two nucleotide sequences, which in turn is based on the fraction of matched complementary nucleotide pairs. The more nucleotides in a given sequence that are complementary to another sequence, the more stringent the conditions can be for hybridization and the more specific will be the binding of the two sequences. Increased stringency is achieved by elevating the temperature, increasing the ratio of co-solvents, lowering the salt concentration, and the like.

The term “complementary,” “complement,” or “complementary nucleic acid sequence” refers to the nucleic acid strand that is related to the base sequence in another nucleic acid strand by the Watson-Crick base-pairing rules. In general, two sequences are complementary when the sequence of one can bind to the sequence of the other in an anti-parallel sense wherein the 3′-end of each sequence binds to the 5′-end of the other sequence and each A, T(U), G, and C of one sequence is then aligned with a T(U), A, C, and G, respectively, of the other sequence. RNA sequences can also include complementary G/U or U/G basepairs.

The term “hybrid” refers to a double-stranded nucleic acid molecule formed by hydrogen bonding between complementary nucleotides. The term “hybridize” refers to the process by which single strands of nucleic acid sequences form double-helical segments through hydrogen bonding between complementary nucleotides.

The term “stringent hybridization conditions” as used herein refers to conditions that are that are compatible to produce duplexes on an array surface between complementary binding members, for example, between probes and complementary targets in a sample, e.g., duplexes of nucleic acid probes, such as DNA probes, and their corresponding nucleic acid targets that are present in the sample, e.g., their corresponding mRNA analytes present in the sample. An example of stringent hybridization conditions is hybridization at 60° C. or higher and 3×SSC (450 mM sodium chloride/45 mM sodium citrate). Another example of stringent hybridization conditions is incubation at 42° C. in a solution containing 30% formamide, 1M NaCl, 0.5% sodium sarcosine, 50 mM MES, pH 6.5. Stringent hybridization conditions are hybridization conditions that are at least as stringent as the above representative conditions, where conditions are considered to be at least as stringent if they are at least about 80% as stringent, typically at least about 90% as stringent as the above specific stringent conditions. Other stringent hybridization conditions are known in the art and may also be employed, as appropriate.

By “remote location,” it is meant a location other than the location at which the array is present and hybridization occurs. For example, a remote location could be another location (e.g., office, lab, etc.) in the same city, another location in a different city, another location in a different state, another location in a different country, etc. As such, when one item is indicated as being “remote” from another, what is meant is that the two items are at least in different rooms or different buildings, and may be at least one mile, ten miles, or at least one hundred miles apart. “Communicating” information references transmitting the data representing that information as electrical signals over a suitable communication channel (e.g., a private or public network). “Forwarding” an item refers to any means of getting that item from one location to the next, whether by physically transporting that item or otherwise (where that is possible) and includes, at least in the case of data, physically transporting a medium carrying the data or communicating the data. An array “package” may be the array plus only a substrate on which the array is deposited, although the package may include other features (such as a housing with a chamber). A “chamber” references an enclosed volume (although a chamber may be accessible through one or more ports). It will also be appreciated that throughout the present application, that words such as “top,” “upper,” and “lower” are used in a relative sense only.

A “computer-based system” refers to the hardware means, software means, and data storage means used to analyze the information of the present invention. The minimum hardware of the computer-based systems of the present invention comprises a central processing unit (CPU), input means, output means, and data storage means. A skilled artisan can readily appreciate that any one of the currently available computer-based system are suitable for use in the present invention. The data storage means may comprise any manufacture comprising a recording of the present information as described above, or a memory access means that can access such a manufacture. The methods or algorithms of the present invention may be carried out using either relatively simple user-written subroutines or publicly available stand-alone software applications. Calculations may be orchestrated and the filtering algorithms may be implemented using any of a number of commercially available computer programs as a framework such as, e.g., Microsoft® Excel spreadsheet, Microsoft® Access relational database, and the like.

To “record” data, programming or other information on a computer readable medium refers to a process for storing information, using any such methods as known in the art. Any convenient data storage structure may be chosen, based on the means used to access the stored information. A variety of data processor programs and formats can be used for storage, e.g. word processing text file, database format, etc.

A “processor” references any hardware and/or software combination that will perform the functions required of it. For example, any processor herein may be a programmable digital microprocessor such as available in the form of an electronic controller, mainframe, server or personal computer (desktop or portable). Where the processor is programmable, suitable programming can be communicated from a remote location to the processor, or previously saved in a computer program product (such as a portable or fixed computer readable storage medium, whether magnetic, optical or solid state device based). For example, a magnetic medium or optical disk may carry the programming, and can be read by a suitable reader communicating with each processor at its corresponding station.

The term “substrate” or “support” refers to a material that has a surface that provides physical support for the features bound thereto. The materials should be of such a composition that they endure the conditions of binding of the features to the surface of the substrate and of any subsequent treatment or handling or processing that may be encountered in the use of the particular substrate. In one embodiment, the substrate material is transparent. By “transparent” is meant that the substrate material permits signal from features on the surface of the substrate to pass therethrough without substantial attenuation and also permits any interrogating radiation to pass therethrough without substantial attenuation. By “without substantial attenuation” may include, for example, without a loss of more than 40% or more preferably without a loss of more than 30%, 20% or 10%, of signal. The interrogating radiation and signal may be, for example, visible, ultraviolet or infrared light. The materials from which the substrate may be fabricated should ideally exhibit a low level of non-specific binding during hybridization events.

The materials may be naturally occurring or synthetic or modified naturally occurring. Suitable rigid substrates may include glass, which term is used to include silica, and include, for example, glass such as glass available as Bioglass, and suitable plastics. Additional rigid, non-transparent materials may be considered, such as silicon, mirrored surfaces, laminates, ceramics, opaque plastics, such as, for example, polymers such as, e.g., poly (vinyl chloride), polyacrylamide, polyacrylate, polyethylene, polypropylene, poly(4-methylbutene), polystyrene, polymethacrylate, poly(ethylene terephthalate), nylon, poly(vinyl butyrate), etc., either used by themselves or in conjunction with other materials. The surface of the substrate is usually the outer portion of a substrate.

The material used for a substrate may take any of a variety of configurations ranging from simple to complex. The support can have any one of a number of shapes, such as strip, plate, disk, rod, particle, including bead, tube, well, and the like. Usually, the material is relatively planar such as, for example, a slide or plate or flat disc and the like. In many embodiments, the material is shaped generally as a rectangular solid. Multiple predetermined arrangements such as, e.g., arrays of capture agents, may be synthesized on a sheet, which is then diced, i.e., cut by breaking along score lines, into single array substrates. Typically, in one embodiment the substrate has a length in the range about 5 mm to 100 cm, usually about 10 mm to 25 cm, more usually about 10 mm to 15 cm, and a width in the range about 4 mm to 25 cm, usually about 4 mm to 10 cm and more usually about 5 mm to 5 cm. The substrate may have a thickness of less than 1 cm, or even less than 5 mm, 2 mm, 1 mm, or in some embodiments even less than 0.5 mm or 0.2 mm. The thickness of the substrate is about 0.01 mm to 5.0 mm, usually from about 0.1 mm to 2 mm and more usually from about 0.2 to 1 mm. The substrate is usually cut into individual test pieces, which may be the size of a standard size microscope slide, usually about 3 inches in length and 1 inch in width.

The substrate surface onto which the polynucleotide compositions or other moieties are deposited may be smooth or substantially planar, or have irregularities, such as depressions or elevations. The surface may be modified with one or more different layers of compounds that serve to modify the properties of the surface in a desirable manner. Such modification layers of interest include: inorganic and organic layers such as metals, metal oxides, polymers, small organic molecules and the like. Polymeric layers of interest include layers of: peptides, proteins, polynucleic acids or mimetics thereof (for example, peptide nucleic acids and the like); polysaccharides, phospholipids, polyurethanes, polyesters, polycarbonates, polyureas, polyamides, polyetheyleneamines, polyarylene sulfides, polysiloxanes, polyimides, polyacetates, and the like, where the polymers may be hetero- or homopolymeric, and may or may not have separate functional moieties attached thereto (for example, conjugated).

The phrase “ancillary materials” refers to various materials that are frequently employed in the methods and assays as discussed herein. For example, buffers and salts will normally be present in an assay medium, as well as stabilizers for the assay medium and the assay components. Frequently, in addition to these additives, proteins may be included, such as albumins, organic solvents such as formamide, quaternary ammonium salts, polycations such as spermine, surfactants, particularly non-ionic surfactants, binding enhancers, e.g., polyalkylene glycols, or the like.

The phrase “evaluating a parameter” refers to determination of the numerical value of a numerical descriptor of a property of an oligonucleotide sequence by means of a formula, algorithm or look-up table.

The term “filter” refers to a mathematical rule or formula that divides a set of numbers into two subsets. Generally, one subset is retained for further analysis while the other is discarded based on a cutoff value.

The phrase “filter set” refers to a set of rules or formulae that successively winnow a set of numbers by identifying and discarding subsets that do not meet specific criteria.

### SPECIFIC EMBODIMENTS

As mentioned above, the present methods are directed to selecting a combination of nucleic acid sample pairs for evaluating the ability of an oligonucleotide probe to measure differential expression of genes in differential gene expression experiments. In the present method differential gene expression experiments are conducted using (i) nucleic acid sample pairs and (ii) nucleic acid probes immobilized on a substrate. The probes represent a set of genes where the number of genes in the set is a portion, and in that sense a predetermined number, of an expected number of genes in a sample. A nucleic acid sample pair combination is selected based on the members of the combination having the most, or second most, or third most, etc., number of genes from the set of genes that exhibit differential expression and the least, or the second least, or the third least, etc., number of the genes that do not exhibit differential expression.

The present methods have application to any situation involving the use of nucleic acid sample pairs particularly in differential gene expression experiments. In one specific application, the present invention may be applied to methods for evaluating probes for their usefulness as surface immobilized probes for a target molecule of interest, e.g., a target nucleic acid. The present invention may be employed in conjunction with methods of identifying or designing probes for use in array structures, where the probes are chemical probes, e.g., biopolymer probes, such as nucleic acids. In particular, the present invention has application to the evaluation of probes for use in gene differential expression analyses. While the following description is provided in terms of nucleic acid probe design protocols for ease and clarity of description, the scope of the invention is not so limited and extends to the identification or design of suitable probes for use in any type of array structure.

In general, differential gene expression assays are typically performed by first providing an array of candidate nucleic acid probes immobilized on a surface of a solid support. The array includes a substrate surface having immobilized thereon a nucleic acid candidate probe for each of the identified candidate probe sequences to be empirically evaluated. In other words, an array is provided that includes a probe for each of the candidate probe sequences to be evaluated, i.e., all of the candidate probe sequences to be evaluated have corresponding probes on the array that include the same sequence. The arrays of candidate probes may be provided in a number of different ways, e.g., via deposition techniques or in situ production, as described in U.S. Pat. Nos. 6,451,998; 6,446,682; 6,440,669; 6,420,180; 6,372,483; 6,323,043; and 6,242,266; the disclosures of which patents are herein incorporated by reference.

The surface immobilized candidate probes having the sequences of the candidate probe sequences are then contacted with two or more sets of nucleic acid sample pairs under differential gene expression analysis conditions to evaluate the probes. In certain embodiments, an identical candidate probe array is contacted with each different sample pair of the set of different sample pairs, while in other embodiments, the same nucleic acid array may be contacted with two or more sample pairs, so long as any hybridized targets from any previous assay are efficiently removed prior to contact with the next sample pair. Protocols for differential gene expression assays are described further below.

The above empirical evaluation process results in the production of a collection of empirically obtained data values for each candidate probe sequence. The empirical data values are measures of performance across a plurality of different experimental sets, specifically a plurality of differential gene expression experiments. In particular, a collection of probe performance data values (e.g., in the form of log ratio values) for each different differential gene expression experiment is obtained for each candidate probe. Accordingly, for each probe an empirical or experimentally determined measure of that probe's performance in each of a number of different differential gene expression assays is obtained. For example, a value is obtained to represent performance of each probe in each experiment. The data making up a given collection of data values may be raw data or processed data and may be a measure of hybridization efficiency, signal intensity, signal log-ratio or combination thereof.

In the aforementioned experiments involving nucleic acid sample pair combinations, it is desirable to have a sample pair combination with maximized diversity so that the sample pair combination has the greatest diversity with respect to genes differentially expressed. Embodiments of the present invention assist in avoiding the selection of nucleic acid sample pairs that have redundant genes considered differentially expressed. In other words, embodiments of the present invention are directed to maximizing the total number of genes differentially expressed in the nucleic acid sample pair selected by the present methods. Embodiments of the present invention may increase the probability of finding a higher number of differentially expressed genes in the nucleic acid sample pair combination. Furthermore, embodiments of the invention may be employed to decrease the number of nucleic acid sample pairs employed in the combination.

The following discussion is directed to the application of the present invention to one approach (referred to herein as the “illustrative method”) for selecting and evaluating probes. This approach is disclosed in U.S. patent application Ser. No. 10/303,160 “Methods For Identifying Suitable Nucleic Acid Probe Sequences For Use In Nucleic Acid Arrays” filed Nov. 22, 2002, by Patrick Collins, et al.) (Collins), the disclosure of which is incorporated herein by reference in its entirety. The application of the present invention to the illustrative method is by way of illustration and not limitation.

In the illustrative method, a set of computationally determined initial candidate sequences are empirically evaluated to obtain functional data. The assumption is that different probes for a given gene will show similar differential expression ratios. The ratios will vary depending on the nucleic acid samples used in the experimental design. However, all of the probes for the same gene should show similar ration values across different tissue combinations. This data is then employed to identify one or more clusters of candidate probe sequences from the initial set such that all candidate probe sequences within each identified cluster exhibit substantially the same performance under a plurality of different experiments, specifically, a plurality of differential gene expression experiments. A candidate probe from the cluster that exhibits the best performance across the plurality of experimental sets is then selected as the optimum candidate probe, e.g., based on one or more performance metrics or parameters. Probes that do not cluster are likely to be cross-hybridizing with other targets and are discarded.

The illustrative method results in the identification of a set or cluster of probes, where the set or cluster of probes are suitable for use as array probes because the selected cluster members exhibit substantially the same performance across a plurality of different experimental sets, specifically a plurality of differential gene expression experiments. A feature of the illustrative method is that it includes both computational steps and empirical steps. Specifically, a collection of candidate probe sequences for a given target nucleic acid is first computationally identified from the sequence of the target nucleic acid of interest. The initially identified candidate sequences are subsequently tested empirically and then further evaluated using additional computational steps in order to identify a suitable set or collection of probe sequences from which “best” probe sequences may be selected.

In a representative example of the above empirical evaluation step of the illustrative method, multiple copies of a microarray that includes candidate 60-mer probes having sequences identified by the prior sequence identification step are produced using an in situ nucleic acid array synthesis protocol. These resultant microarrays are then hybridized to 10 different nucleic acid sample pair (tissue/cell line) combinations (e.g., 4 replicates per sample pair): one self vs. self and 9 sample pairs chosen to maximize the number of mRNA's that are differentially expressed between the members of the pair. Thus, in this approach, the probe validation process relies on the ability of 10 different probes per gene to cluster together across 9 different nucleic acid sample pair combinations (plus one self vs. self experiment to assess dye label bias). As mentioned above, the nucleic acid sample pair combination selected should have as many expressed genes as possible and as many differentially expressed genes as possible. Ideally, 100% of the genes tested should show differential expression in at least 1 out of the 9 sample pairs tested. Although the illustrative method discussed above employs 9 different nucleic acid sample pairs, this is by way of example. The number of nucleic acid sample pairs employed in the illustrative probe validation method may be about 5 to about 15, about 8 to about 10, and so forth.

In accordance with the present invention, probes representing a relatively small set of genes may be employed to optimize the selection of the nucleic acid sample pairs that will be used in the illustrative probe validation method discussed above from a large pool of nucleic acid sample pairs. Typically, in the present methods about 5 to about 50 or more, about 10 to about 40, about 15 to about 30, for example, about 20, nucleic acid sample pairs are screened. The nucleic acid sample pairs are chosen based on considerations such as, for example, availability, price, theoretical degree of differential expression and so forth. The members of the nucleic acid sample pairs usually comprise a label where the label for one of the members is different from the label of the other member. For example, one member may comprise a fluorescent dye label and the other member may comprise a different fluorescent dye label.

The genes are randomly selected or selected based on their predicted expression levels and so forth. The number of genes for which probes are employed in the present method is, in general, that which is sufficient to achieve maximization of differential expression. A relatively small number or predetermined number of genes is utilized in order to reduce the amount of time and materials employed to achieve the benefits of the present methods. Typically, the number of genes for which probes are employed in the present method is about 50 to about 5000, about 100 to about 4000, about 500 to about 3000, or about 1500 to about 2500.

A plurality of probes is employed for each gene selected. Each of the probes for a particular gene binds with a different sequence of the gene. The probes for each gene are selected based on probe design algorithms (see, for example, Collins, supra). The probes are bound to a surface of a substrate by techniques that are well known in the art as mentioned above. For example, probes may be deposited dropwise on to a surface of a substrate that has been activated to react with the gene to bind the gene to the surface to form an array of genes on the surface. On the other hand, the probes may be synthesized in situ on the surface of the substrate as mentioned above. In some embodiments, the probes are in the form of an addressable array on the surface of the substrate.

In the next step of an embodiment of a method of the invention, the surface of the substrate comprising the plurality of probes is exposed to each pair of the nucleic acid sample pairs and hybridization results are determined. To this end, the surface of the substrate is exposed to the nucleic acid sample pair and to other reagents such as ancillary materials and reagents for carrying out the hybridization reactions. The methods of the invention may be carried out with multiple substrates with the same plurality of probes where each substrate is used for a different nucleic acid sample pair. On the other hand, the same substrate with the surface comprising a plurality of probes may be employed sequentially with a different nucleic acid sample pair as long as the prior samples are sufficiently removed from the surface of the substrate prior to contact with a subsequent sample pair. Sufficient removal is usually achieved by washing with an appropriate removal buffer until the array may be employed with another sample pair in a meaningful manner without interference from the previous sample pair.

Alternatively, the two members of a sample pair may be separately contacted with a separate array of candidate probes to conduct hybridization experiments. The data obtained from the separate experiments may be used in a manner similar to the data obtained from conducting the hybridization using the combined members of the nucleic acid sample pair.

The substrate surface exposed to the nucleic acid sample pair, or to the individual members of the nucleic acid sample, is incubated under conditions suitable for hybridization reactions to occur. Typically, a buffer solution is employed and the nucleic acid sample is allowed to hybridize to the arrayed genes according to methods known in the art. The hybridization conditions include appropriate time, concentrations, temperature and the like for hybridization reactions to occur. The exact hybridization conditions will depend upon the nature of the nucleic acid sample pairs, the nature of the genes on the surface, and the like. In some instances stringent hybridization conditions are employed.

After the appropriate period of time of contact between the nucleic acid sample pairs, or the individual members thereof, and the probes on the surface of the substrate, the contact is discontinued and various processing steps are performed. Following the processing of the substrate, it is moved to an examining device where the surface of the substrate on which the arrays are disposed is interrogated. The examining device may be a scanning device involving an optical system.

In the present method an estimation is made for each gene in each nucleic acid pair experiment whether the gene is differentially expressed and whether probes for that gene have the potential to cluster together to enable the probe validation algorithm to select an optimal probe. Whether or not a particular gene in a given nucleic acid pair experiment falls into the above category may be based on one or more performance metrics. The performance metrics include, among others, the following: a) the probability of the combined LogRatio (across all the replicates) being significantly different from zero (P-value) for each of the probes representing a gene and b) the number of probes that represent a given gene that have a probability of the combined LogRatio value being significantly different from zero above a given threshold. One or more parameters other than LogRatio may be utilized in combination with LogRatio or P-value or in some cases independent of LogRatio or P-value. These parameters include, for example, LogRatio error, signal intensities in the green (from a particular fluorescent label on one of the member of the nucleic acid sample pair) and red (from a particular fluorescent label on the other member of the nucleic acid sample pair) channels, quality of the probes typically based on specificity, and so forth.

Typically, LogRatio is employed and, if the number of probes indicating differential expression is greater than desired based on experimental considerations and the like, one or more of the other parameters may be employed in combination with LogRatio. For each additional parameter, cutoff ranges are established. These ranges may be adjusted larger or smaller based on the aforementioned considerations regarding the number of probes indicating differential expression.

The arrays are then scanned, as described in greater detail below, and the probe or feature data are extracted using extraction software, such as, for example, Agilent's Feature Extraction software (available from Agilent Technologies, Palo Alto, Calif.). In one specific embodiment, confidence measure or level of confidence of an observed expression value may be employed. Typically, the feature extraction protocol computes P-values, specifically the likelihood that the LogRatio is significantly different from 0. The feature data are further processed to exclude data from features that do not satisfy certain quality control measures, e.g., signal saturation or the presence of too many outlier pixel values and to exclude data from probes that do not generate sufficient signal in any of the experiments. The obtained feature data are further processed by combining replicate experiments using statistical weights derived from the P-values associated with each feature, e.g., by using a processing algorithm designed for this purpose.

For example, a confidence level for a P-value may be set at 0.0001. This cutoff level is used to evaluate the probes. Only probes with a P-value that is 0.0001 or less are considered as representing a gene that is differentially expressed. This empirical data may be evaluated as to whether the values meet or exceed a minimum performance metric or cutoff value. The number of probes exhibiting the aforementioned P-value is also used for a cutoff level. Thus, the cutoff level for the number of probes exhibiting the above P-value may be set at, for example, 4. If 4 out of a total of 10 probes per gene exhibit the above P-value, then the gene is considered differentially expressed. Otherwise, the gene is not considered differentially expressed. The cutoff levels for the P-value and the number of probes exhibiting such a P-value may be adjusted accordingly in order to obtain a significant number of genes for the next step of the process.

For a gene that exhibits differential expression for a nucleic acid sample pair, a “yes” value is assigned, and for each gene that does not exhibit differential expression for a nucleic acid sample pair, “no” value is assigned. The above steps are repeated for the number of nucleic acid sample pairs to be evaluated. The data is tabulated for each combination of nucleic acid sample pairs to be evaluated. A combination of nucleic acid sample pairs is selected having a score based on a maximized number of “yes's” and a minimized number of “no's.” In one approach, if a gene is considered differentially expressed and could be successfully used in a probe validation assay involving differential expression experiments, a score of 1 is assigned to that gene. Otherwise, the gene is assigned a score of 0. The assignment of a 1 or a 0 representing yes and no, respectively, is conveniently employed where embodiments of the present methods are carried out with the aid of a computer. A table is built with values for all the genes in the test arrays across all the nucleic acid sample pairs studied.

An analysis is carried out for all of the possible combinations having a predetermined number “n” of nucleic acid sample pairs out of the total number of sample pairs employed. In a first step, the sum of the values for every gene across the predetermined number of nucleic acid sample pairs is ascertained. If the gene is not considered suitable for the probe validation process in any of the nucleic acid sample pairs, the sum is zero. In contrast, if a gene is appropriate for the probe validation process in all nucleic acid sample pairs, the sum of the values is equivalent to the predetermined number “n” of nucleic acid sample pairs. Thus, each gene for each of the nucleic acid sample combinations having n number of nucleic acid sample pairs has a final value between 0 and n.

The above will be explained in more detail using the following example by way of illustration and not limitation. For purposes of this example, the nucleic acid sample pairs are tissue pairs. The total number of tissue pairs to be evaluated is 19 and only one combination of 9 (n=9) tissue pairs will be selected out of all of the possible combinations of 9 tissue pairs out of the total of 19. The tissue pairs are designated TP1, TP2, TP3, TP4, TP5, TP6, TP7, TP8, TP9, TP10, TP11, TP12, TP13, TP14, TP15, TP16, TP17, TP18 and TP19. A computer program may be employed that facilitates the above analysis. The number of genes for which probes are employed in an array on the surface of the substrate is 2000. The genes are designated Gene 1, Gene 2, Gene 3, . . . Gene 2000. Ten probes per gene are employed. One tissue pair member is labeled with a red dye and the other member of the tissue pair is labeled with a green dye. Hybridization experiments are conducted as discussed above, the results are analyzed, and the data is recorded using appropriate software and computer hardware. If a gene is not considered suitable for the probe validation process in any of the tissue pairs based on the data obtained using the aforementioned cutoff levels, the sum of the values assigned as discussed above would be 0. In contrast, if a gene is appropriate for the probe validation process in all tissue pairs, the sum of the values would be 9. Thus, each gene, in each of the 9 tissue pair combinations, will have a value between 0 and 9.

Using the results obtained in the above experiments, a set of tables is constructed. Each table of the set has rows representing the 9 tissue pairs of the combination evaluated and has columns representing the genes evaluated using the probes as discussed above. The set of tables comprises such a table constructed for each of the tissue pair combinations tested. An example of a portion of one such a table for tissue pair combination TP1, TP2, TP3, TP4, TP5, TP6, TP7, TP8, and TP9 is as follows:

| | Tissue Pair | | | | | | | | | |
|---|---|---|---|---|---|---|---|---|---|---|
| Gene # | TP1 | TP2 | TP3 | TP4 | TP5 | TP6 | TP7 | TP8 | TP9 | Total |
| Gene 1 | 0 | 0 | 0 | 0 | 0 | 0 | 0 | 0 | 0 | 0 |
| Gene 2 | 1 | 1 | 1 | 1 | 1 | 1 | 1 | 1 | 1 | 9 |
| Gene 3 | 1 | 0 | 0 | 0 | 0 | 1 | 1 | 0 | 1 | 4 |
| 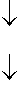 | | | | | | | | | | |
| Gene 2000 | 1 | 0 | 0 | 0 | 0 | 0 | 1 | 0 | 0 | 2 |

An example of a portion of another such a table for tissue pair combination TP1, TP3, TP4, TP5, TP6, TP7, TP8, TP9 and TP10 is as follows:

| | Tissue Pair | | | | | | | | | |
|---|---|---|---|---|---|---|---|---|---|---|
| Gene # | TP1 | TP3 | TP4 | TP5 | TP6 | TP7 | TP8 | TP9 | TP10 | Total |
| Gene 1 | 0 | 0 | 0 | 0 | 0 | 0 | 0 | 0 | 1 | 1 |
| Gene 2 | 1 | 1 | 1 | 1 | 1 | 1 | 1 | 1 | 0 | 8 |
| Gene 3 | 1 | 0 | 0 | 0 | 1 | 1 | 0 | 1 | 1 | 5 |
| 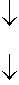 | | | | | | | | | | |
| Gene 2000 | 1 | 0 | 0 | 0 | 0 | 0 | 1 | 0 | 1 | 3 |

An example of a portion of another such a table for tissue pair combination TP1, TP4, TP5, TP6, TP7, TP8, TP9, TP10, and TP11 is as follows:

| | Tissue Pair | | | | | | | | | |
|---|---|---|---|---|---|---|---|---|---|---|
| Gene # | TP1 | TP4 | TP5 | TP6 | TP7 | TP8 | TP9 | TP10 | TP11 | Total |
| Gene 1 | 0 | 0 | 0 | 1 | 0 | 0 | 0 | 1 | 1 | 3 |
| Gene 2 | 1 | 1 | 1 | 1 | 1 | 1 | 1 | 1 | 1 | 9 |
| Gene 3 | 1 | 0 | 0 | 1 | 1 | 0 | 1 | 1 | 1 | 6 |
| 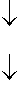 | | | | | | | | | | |
| Gene 2000 | 1 | 0 | 0 | 0 | 0 | 1 | 0 | 1 | 0 | 3 |

The data obtained and tabulated as above as set forth in the above set of tables are used to prepare a second table comprising, for example, rows representing the totals (Number Totals) for the number of 0's, 1's, 2's, 3's, 4's, 5's, 6's, 7's, 8's and 9's obtained for each of the tissue pair combinations evaluated and comprising columns representing the tissue pair combinations. The designations “row” and “column” are arbitrary and the data may be organized in any configuration so long as a totaling of the aforementioned numbers may be achieved.

| | Number Totals | | | | | | | | | |
|---|---|---|---|---|---|---|---|---|---|---|
| Tissue Pair Combination | 0 | 1 | 2 | 3 | 4 | 5 | 6 | 7 | 8 | 9 |
| 1,2,3,4,5,6,7,8,9 | 407 | 133 | 102 | 96 | 128 | 173 | 259 | 347 | 314 | 191 |
| 1,3,4,5,6,7,8,9,10 | 408 | 158 | 103 | 98 | 125 | 169 | 254 | 317 | 328 | 190 |
| 1,4,5,6,7,8,9,10,11 | 604 | 49 | 39 | 74 | 147 | 214 | 312 | 315 | 264 | 132 |
| 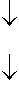 | | | | | | | | | | |
| 11,12,13,14,15,16,17,18,19 | 568 | 122 | 54 | 19 | 56 | 432 | 157 | 369 | 163 | 103 |

Following the tabulation of the results of the differential expression experiments as discussed above, a tissue pair combination is selected for use in differential gene expression experiments for the evaluation of probes for a particular array of probes on a surface of a substrate. The tissue pair combination selected has a high probability of finding a higher number of differentially expressed genes in differential expression experiments. In the above method, the total scores are observed for the number of 9's that a particular tissue pair combination received and for the number of 0's received. The tissue pair combination exhibiting the highest score for the number of 9's and the lowest score for the number of 0's is the tissue pair combination chosen and represents the tissue pair combination that achieves the for validating probes for their use in analyses involving differential gene expression. In the above example, tissue pair combination TP1, TP2, TP3, TP4, TP5, TP6, TP7, TP8, and TP9 has a score of 191 for number of 9's and 407 for the number of 0's. This tissue pair combination, therefore, exhibits the highest score for the number of 9's and the lowest score for the number of 0's.

It should be noted that in the above example, tissue pair combination TP1, TP3, TP4, TP5, TP6, TP7, TP8, TP9 and TP10 has a score of 190 for number of 9's and 408 for the number of 0's. This tissue pair combination, therefore, exhibits the second highest score for the number of 9's and the second lowest score for the number of 0's. This tissue pair combination is considered to be of interest in conducting differential expression experiments and might be chosen if the tissue pair combination having the highest score for number of 9's and the lowest score for the number of 0's is not suitable for a particular differential expression experiment for some other reason. Accordingly, a combination of nucleic acid sample pairs may be selected based on a score representing a maximized number of n's (9's in this example) and a minimized number of 0's. Thus, the nucleic acid sample pair ultimately selected may have the greatest number of n's, the second greatest number of n's, third greatest number of n's and so forth and the least number of 0's, the second least number of 0's, the third least number of 0's, and so forth. In this way the selection is made on a maximized number and a minimized number, which means that the selected pair may exhibit the maximum number of n's or the next to maximum number of n's or the next to next maximum number of n's and so forth and that the selected pair may exhibit the minimum number of 0's, the next to minimum number of 0's, the next to next minimum number of 0's and so forth.

The results of the above analysis in accordance with the present invention can also be employed to decrease the number of tissue pairs employed in a particular tissue pair combination used in differential expression experiments. In this aspect of the present methods, tissue pairs may be eliminated by setting a minimum value for the percentage of genes that must be differentially expressed in a given combination. The number of tissue pairs that are needed is that which is necessary to reach that minimum. Of course, additional tissue pairs may be employed if desired.

The aforementioned methods of the present invention are preferably carried out at least in part with the aid of a computer system. For example, an IBM® compatible personal computer (PC) may be utilized. The computer is driven by software specific to the methods described herein.

Another aspect of the present invention is a computer-based method for selecting a combination of nucleic acid sample pairs for evaluating the ability of an oligonucleotide probe to measure differential expression of genes. The method comprises: (a) contacting a nucleic acid sample pair from a plurality of nucleic acid sample pairs with a plurality of probes for each of a predetermined number of genes to determine whether the genes exhibit differential expression, (b) under computer control, determining for each gene and each of the nucleic acid sample pairs whether the gene exhibits differential expression based on one or more parameters, (c) under computer control, for each gene determining a score representing the total number of probes exhibiting differential expression, for example, assigning a “yes” value for a gene that exhibits differential expression for a nucleic acid sample pair and a “no” value for each gene that does not exhibit differential expression for a nucleic acid sample pair. (d) repeating steps (a)-(d) for a desired number of nucleic acid sample pairs from the plurality of nucleic acid sample pairs, (e) under computer control, tabulating the data obtained, and (f) under computer control, selecting a combination of nucleic acid sample pairs having a score based on a maximized number of “yes's” and a minimized number of “no's.”

The results obtained by contacting the arrays of probes with nucleic acid sample pairs may be analyzed utilizing an analysis system comprising a computer. The computer comprises: (i) means for assigning, for each gene and to each of the nucleic acid sample pairs, a value of 1 (representing a “yes”) if the gene exhibits differential expression or assigning a value of 0 (representing a “no”) if the gene does not exhibit differential expression, (ii) means for tabulating data from step (i) to determine the total number scores for each combination of the nucleic acid sample pairs to be evaluated, wherein the maximum number of 1's for each nucleic acid sample pair per gene is the number “n” of nucleic acid sample pairs of the combination, and (iii) means for selecting a combination of nucleic acid sample pairs based on a score representing a maximized number of n's and a minimized number of 0's.

A computer program and computer are utilized to carry out the above method steps. The computer program provides for (i) input of data from the reading of the hybridization experiments involving the array of probes and the nucleic acid sample pairs, (ii) efficient algorithms for the aforementioned means for assigning, means for determining, means for tabulating and means for selecting, (iii) efficient, versatile mechanisms for filtering the aforementioned data, (iv) mechanisms for computation involving the aforementioned data, and (v) mechanisms for outputting the final results in a versatile, machine-readable or human-readable form.

The computer system may be programmed from a computer readable storage medium that carries code for the system to execute the steps required of it, thus, having programming stored thereon for implementing the subject methods. The computer readable media may be, for example, in the form of a computer disk or CD, a floppy disc, a magnetic “hard card”, a server, or any other computer readable media capable of containing data or the like, stored electronically, magnetically or optically and including, for example, machine readable bar code, solid state electronic storage devices such as random access memory (RAM), or read only memory (ROM), or any other physical device or medium that might be employed to store a computer program. It will also be understood that computer systems of the present invention can include the foregoing programmable systems and/or hardware or hardware/software combinations that can execute the same or equivalent steps. Accordingly, stored programming embodying steps for carrying-out the subject methods may be transferred to a computer such as a personal computer (PC), (i.e., accessible by a researcher or the like), by physical transfer of a CD, floppy disk, or like medium, or may be transferred using a computer network, server, or other interface connection, e.g., the Internet.

In one embodiment of the subject invention, a system of the invention may include a single computer or the like with a stored algorithm capable of carrying out suitable probe identification methods, i.e., a computational analysis system. In certain embodiments, the system is further characterized in that it provides a user interface, where the user interface presents to a user the option of selecting among one or more different, including multiple different, inputs, e.g., various parameter values for the algorithm, as described above. Computational systems that may be readily modified to become systems of the subject invention include those described in U.S. Pat. No. 6,251,588; the disclosure of which is herein incorporated by reference.

Arrays

The above-described methods and devices programmed to practice the same may be used to identify probe nucleic acids to be produced on surfaces of any of a variety of different substrates, including both flexible and rigid substrates, e.g., in the production of nucleic acid arrays. Such arrays may be prepared by methods such as the in situ and deposition methods referred to above.

A feature of the subject arrays is that they include one or more, usually a plurality of, probes whose sequence as been selected according to the protocols that involve nucleic acid sample pairs identified by methods of the present invention. Because the sequences of the probes on the arrays are selected according to the above protocols, the probe sequences are ones that exhibit substantially similar performance to other probes for the same gene under a plurality of different differential gene expression protocols. For example, the probe sequences on the array will provide performance that varies little, if any, to a plurality of other probes to the same gene between two or more different differential gene expression assays, i.e., it performs substantially similar to other probes for the same gene under a plurality of different experimental conditions. Where the performance parameter used to determine similarity is hybridization efficiency, the magnitude of any difference observed in hybridization efficiency between among different probes for the same gene and across any two different differential gene expression analysis protocols does not vary by more than about 15-fold, and usually by not more than about 10-fold in certain embodiments. In addition, the subject probes of the arrays identified by the subject methods are ones that provide for high specificity and sensitivity, as described above. In many embodiments, at least about 25 number %, such as at least about 50 number %, 75 number % or more, e.g., 90, 95 or 99 or more, up to an including 100 number %, of the probes of the array are probes identified by the subject methods.

Each distinct nucleic acid sequence of the array is typically present as a composition of multiple copies of the polymer on the substrate surface, e.g. as a spot on the surface of the substrate. The number of distinct nucleic acid sequences, and hence spots or similar structures (i.e., array features), present on the array may vary, but is at least 2, at least 10, at least 100, where the number of different spots on the array may be as high as 500, as high as 1,000, as high as 5,000, as high as 10,000, as high as 100,000 or higher, depending on the intended use of the array. The spots of distinct nucleic acids present on the array surface are generally present as a pattern, where the pattern may be in the form of organized rows and columns of spots, e.g., a grid of spots, across the substrate surface, a series of curvilinear rows across the substrate surface, e.g., a series of concentric circles or semi-circles of spots, and the like. The density of spots present on the array surface may vary, but will generally be at least about 10 and usually at least about 100 spots/cm², where the density may be as high as 10⁶ or higher, but will generally not exceed about 10⁵ spots/cm². In the subject arrays of nucleic acids, the nucleic acids may be covalently attached to the arrays at any point along the nucleic acid chain, but are generally attached at one of their termini, e.g., the 3′ or 5′ terminus.

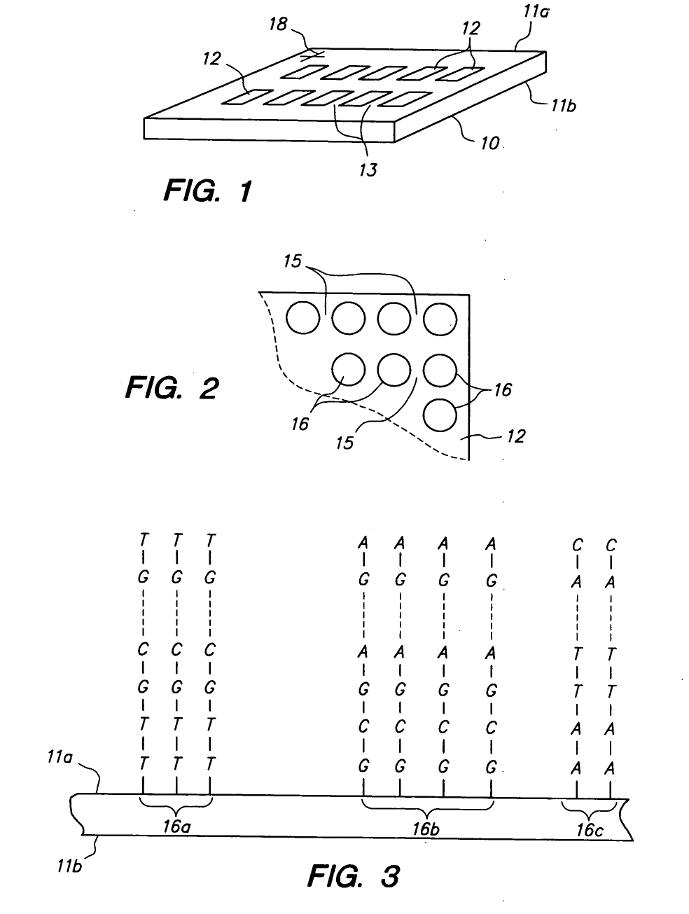

Referring FIGS. 1-3, an example is shown of multiple identical arrays 12 (only some of which are shown in FIG. 1), separated by inter-array regions 13, across the complete front surface 11a of a single transparent substrate 10. However, the arrays 12 produced on a given substrate need not be identical and some or all could be different. Each array 12 will contain multiple spots or features 16 separated by inter-feature regions 15. A typical array 12 may contain from 100 to 100,000 features. All of the features 16 may be different, or some or all could be the same. Each feature carries a predetermined moiety (such as a particular polynucleotide sequence), or a predetermined mixture of moieties (such as a mixture of particular polynucleotides). This is illustrated schematically in FIG. 3 where different regions 16 are shown as carrying different polynucleotide sequences. Arrays of FIGS. 1-3 can be manufactured by in situ or deposition methods as discussed herein.

The subject arrays find use in a variety applications, where such applications are generally analyte detection applications in which the presence of a particular analyte in a given sample is detected at least qualitatively, if not quantitatively. Protocols for carrying out such assays are well known to those of skill in the art and need not be described in great detail here. Generally, the sample suspected of comprising the analyte of interest is contacted with an array produced according to the subject methods under conditions sufficient for the analyte to bind to its respective binding pair member that is present on the array. Thus, if the analyte of interest is present in the sample, it binds to the array at the site of its complementary binding member and a complex is formed on the array surface. The presence of this binding complex on the array surface is then detected, e.g. through use of a signal producing system, e.g., an isotopic or fluorescent label present on the analyte, etc. The presence of the analyte in the sample is then deduced from the detection of binding complexes on the substrate surface.

Specific analyte detection applications of interest include hybridization assays in which the nucleic acid arrays of the subject invention are employed. In these assays, a sample of target nucleic acids is first prepared, where preparation may include labeling of the target nucleic acids with a label, e.g., a member of signal producing system. In some embodiments, a collection of labeled control targets may be included in the sample, where the collection may be made up of control targets that are all labeled with the same label or two or more sets that are distinguishably labeled with different labels, as described above. Following sample preparation, the sample is contacted with the array under hybridization conditions, whereby complexes are formed between target nucleic acids that are complementary to probe sequences attached to the array surface. The presence of hybridized complexes is then detected. Specific hybridization assays of interest that may be practiced using the subject arrays include: gene discovery assays, differential gene expression analysis assays, nucleic acid sequencing assays, and the like.

Kits

Another aspect of the present invention relates to kits useful for conveniently performing a method in accordance with the invention. In one embodiment the kit provides for selecting a combination of nucleic acid sample pairs for evaluating the ability of an oligonucleotide probe to measure differential expression of genes. The kit comprises (a) an algorithm for use in conducting differential gene expression experiments using (i) nucleic acid sample pair combinations representing a predetermined number of nucleic acid sample pairs and (ii) nucleic acid probes immobilized on a substrate where the probes represent a set of genes. The number of genes in the set is a portion of an expected number of genes in a sample. The algorithm is present on a computer readable medium. The kit also includes instructions for using the algorithm to select a nucleic acid sample pair combination based on the members of the combination having a maximized number, e.g., the most, second most, etc., number, of genes from the set of genes that exhibit differential expression and a minimized number, e.g., the least, the second least, etc., number, of the genes that do not exhibit differential expression.

Another embodiment of a kit in accordance with the present invention comprises an algorithm that provides for assigning, for each gene and to each of the nucleic acid sample pairs, a value of 1 (representing a “yes”) if the gene exhibits differential expression or assigning a value of 0 (representing a “no”) if the gene does not exhibit differential expression, (ii) for tabulating data from step (i) to determine the total number scores for each combination of the nucleic acid sample pairs to be evaluated, wherein the maximum number of 1's for each nucleic acid sample pair per gene is the number “n” of nucleic acid sample pairs of the combination, and for selecting a combination of nucleic acid sample pairs based on a score representing a maximized number of n's and a minimized number of 0's.

Another embodiment of a kit in accordance with the present invention is a kit for identifying a sequence of a nucleic acid that is suitable for use as a substrate surface immobilized probe for a target nucleic acid. The kit comprises (a) an algorithm that identifies a sequence of a nucleic acid that is suitable for use as a substrate surface immobilized probe for the target nucleic acid according to the method of the present invention applied to the selection of probes using differential gene expression analysis, wherein the algorithm is present on a computer readable medium; and (b) instructions for using the algorithm to identify the sequence of a nucleic acid that is suitable for use as a substrate surface immobilized probe for the target nucleic acid.

Kits for use in analyte detection assays are also provided. The kits at least include the arrays prepared from probes identified utilizing nucleic acid sample pairs identified in accordance with the present invention, as described above. The kits may further include one or more ancillary materials and additional components necessary for carrying out an analyte detection assay, such as sample preparation reagents, buffers, labels, and the like. As such, the kits may include one or more containers such as vials or bottles, with each container containing a separate component for the assay, and reagents for carrying out an array assay such as a nucleic acid hybridization assay or the like. The kits may also include a denaturation reagent for denaturing the analyte, buffers such as hybridization buffers, wash mediums, enzyme substrates, reagents for generating a labeled target sample such as a labeled target nucleic acid sample, negative and positive controls and written instructions for using the array assay devices for carrying out an array based assay. Such kits also typically include instructions for use in practicing array-based assays.

Kits for use in connection with the probe design protocols may also be provided. Such kits preferably include at least a computer readable medium including programming as discussed above and instructions. The instructions may include installation or setup directions. The instructions may include directions for use of the invention.

Providing software and instructions as a kit may serve a number of purposes. The combinations may be packaged and purchased as a means of upgrading an existing fabrication device. Alternatively, the combination may be provided in connection with a new device for fabricating arrays, in which the software may be preloaded on the same. In which case, the instructions will serve as a reference manual (or a part thereof) and the computer readable medium as a backup copy to the preloaded utility.

The instructions of the above-described kits are generally recorded on a suitable recording medium. For example, the instructions may be printed on a substrate, such as paper or plastic, etc. As such, the instructions may be present in the kits as a package insert, in the labeling of the container of the kit or components thereof (i.e. associated with the packaging or sub packaging), etc. In other embodiments, the instructions are present as an electronic storage data file present on a suitable computer readable storage medium, e.g., CD-ROM, diskette, etc, including the same medium on which the program is presented.

In yet other embodiments, the instructions are not themselves present in the kit, but means for obtaining the instructions from a remote source, e.g. via the Internet, are provided. An example of this embodiment is a kit that includes a web address where the instructions can be viewed and/or from which the instructions can be downloaded. Conversely, means may be provided for obtaining the subject programming from a remote source, such as by providing a web address. Still further, the kit may be one in which both the instructions and software are obtained or downloaded from a remote source, as in the Internet or World Wide Web. Some form of access security or identification protocol may be used to limit access to those entitled to use the subject invention. As with the instructions, the means for obtaining the instructions and/or programming is generally recorded on a suitable recording medium.

Illustrative Method

The following discussion briefly describes the illustrative method (mentioned above) to which the present invention may be applied. It is important to note that some of the discussion below may have application to the present methods particularly as it applies to the selection of probes for immobilizing on the surface of a substrate to conduct the present methods. The illustrative method includes the following steps:
(a) identifying a plurality of candidate probe sequences for the target nucleic acid;
(b) empirically evaluating each of the identified candidate probe sequences;
(c) clustering the identified candidate probe sequences into two or more groups of candidate probe sequences based observed empirical data values, where clustered members exhibit substantially the same performance across a plurality of different experimental sets, specifically a plurality of differential gene expression experiments;
(d) selecting one of the two or more groups of candidate probe sequences as the “best” group; and
(e) choosing a candidate probe sequence from the selected “best” group as the sequence that is most suitable for use as a probe for the target nucleic acid of interest.

As mentioned above, the first step in the illustrative method is to identify a plurality of candidate probe sequences for a given target nucleic acid of interest. The target nucleic acid of interest is generally a nucleic acid of known sequence, where the length of the nucleic acid may vary, but typically ranges from about 200 nt to about 4,000 nt, such as from about 400 nt to about 2,500 nt, including from about 800 nt to about 1,500 nt. In many embodiments, the target nucleic acid has the sequence of an mRNA transcript of interest or the complementary sequence thereof, or the sequence of a first or second strand DNA prepared from an mRNA of interest.

The candidate probes are identified based on a least one selection criterion, wherein in many embodiments a plurality of different selection criteria are employed together to identify the candidate probes from the target nucleic acid sequence. By “plurality” is meant at least about 2, and may be as great as 10 or more, but is typically less than 5, e.g., 2 to 3.

One selection criterion of interest that may be employed is distance from the 3′-end of the in RNA transcript that corresponds to the target nucleic acid, e.g., that is the target nucleic acid or is the complement of the target nucleic acid, or from which the target nucleic acid is derived, e.g., where the target nucleic acid is first or second strand cDNA. When this criterion is employed, candidate sequences of the target nucleic acid are chosen that are within at least about 2,000 nucleotides (nt), within about 1,500 nt, within about 800 nt, of the 3′ end of the mRNA that corresponds to the target nucleic acid.

Another selection criterion that may be employed in the illustrative method is the base composition of the probe sequence. When this criterion is employed, sequences that are abnormally GC rich or poor, long runs of a single base, and/or base compositions that are known to generate unacceptable array features, e.g., under in situ production conditions, are avoided. Sequences that are abnormally GC rich or poor are those sequences whose number % of G and C bases are greater than about 30, such as greater than about 35, or less than about 60, such as less than about 45. By “long run” of a single base is meant a stretch of nucleotides of the same base that is greater than about 6, such as greater than about 10. Sequences that are known to generate unacceptable array features include, but are not limited to, those containing stretches of at least 10 G's.

Another selection criterion employed in the illustrative method is homology of the candidate probe sequence to other sequences from the same organism, i.e., to other mRNA transcripts or complements thereof of the same organism from which the target sequence of interest for which the probe is being designed is obtained. Sequences with a high potential to hybridize to more than one mRNA transcript from a given organism are avoided. Cross-hybridization potential of candidate sequences may be estimated via thermodynamic scoring of the output of BLAST, a standard bioinformatics application used to detect sequence homology and well known to those of skill in the art, or any other convenient cross-hybridization potential assessment protocol. Use of this criterion results in the identification of probe sequences that are specific for the target nucleic acid of interest.

In certain embodiments of the illustrative method, the identification process or algorithm that is employed is one in which parameters are used that minimize the number of identified candidate probe sequences that overlap with each other. Any of the above listed criteria may be adjusted in order to result in minimal overlap of the identified candidate probe sequences. The overlap parameter is designed to yield candidate probes that span the target. If it is not specified, the algorithm employed may identify probes that are heavily overlapped (up to 59 out of 60 bases). While these may be the best probes, using such a set of candidates confounds the clustering analysis, since almost by definition such probes will cluster tightly.

Using the above protocol, a plurality of candidate probe sequences are identified for a given target nucleic acid. In many embodiments of the illustrative method, the number of identified candidate probe nucleic acid sequences is at least about 5, usually at least about 7 and may be as great as 15, 20 or more, but typically does not exceed about 15, where in certain embodiments, the number of candidate probe sequences identified for a given target nucleic acid ranges from about 7 to 12, e.g., 8, 9, 10 or 11.

In certain embodiments of the illustrative method, an algorithm is employed, e.g., in conjunction with a computational analysis system, to identify candidate probe sequences from a target nucleic acid. Any convenient algorithm or process capable of performing the above function may be employed. Of interest are the Agilent probe design algorithms (Agilent Technologies, Palo Alto, Calif.), where the algorithms are employed in identification of candidate probe sequences. Specifically, the design parameters that may be employed include: a) the preferred and allowed distances from the 3′ end, b) the number of probes required before ending base composition iteration (where a suitable number typically ranges from about 20 to about 200, usually from about 50 to about 100), c) the criteria used to label probes as “overlap” (where “overlap” may be defined as probes whose sequences overlap by a number of bases, for example, greater than 10 nt, more typically greater than 40 nt), and d) the number of probes required before the homology calculation (where a suitable number typically ranges from about 10 to about 40, from about 12 to about 20).

As indicated above, the above first step in the illustrative method results in the identification of a plurality of different candidate probe sequences for a given target nucleic acid.

In the next step of the illustrative method, each of the identified candidate probe sequences is evaluated empirically. Specifically, each of the identified candidate probe sequences is evaluated for their performance under a plurality of different experimental sets, specifically a plurality of differential gene expression experiments to obtain a collection of empirically obtained performance data values for each of the candidate nucleic acid probe sequences for each of the plurality of different experimental conditions. In embodiments of the illustrative method, the experimental conditions are differential gene expression assay experiments, where a given experimental condition is a differential gene expression assay using a particular nucleic acid sample pair. Each sample of the pair is obtained from a different source, e.g., tissue or cell line. Differential gene expression array based assays are well known to those of skill in the art. The number of different differential gene expression array based assays for which a given candidate probe is empirically evaluated may vary. The number may range from about 2 to about 20, such as from about 5 to about 15, including from about 7 to about 12, e.g., 10. Any two differential gene expression assays or protocols are considered different if at least one of the nucleic acid samples making up the pairs of any two pairs differs between the two pairs.

In the next step of the illustrative method, the candidate probe sequences are clustered into two or more groups of candidate probe sequences. The candidate probe sequences are divided into two or more groups of candidate probe sequences based on the observed empirical data values obtained in the prior empirical evaluation step.

In many embodiments of this clustering or grouping step, an expression vector for each of the candidate probe sequences is obtained first using the candidate probe sequence's collection of empirical data values. From the obtained expression vector for each candidate probe sequence, a similarity matrix is derived for the set of the candidate probe sequences. The similarity matrix provides a measure of the similarity of the functions of the candidate probe sequence as compared to the other candidate probe sequences being evaluated. Based on the derived similarity matrix for the set of candidate probe sequences, the candidate probe sequences are then grouped into two or more groups.

The first substep of the clustering step is the generation of an expression vector for each candidate probe sequence, where the expression vector is generated using the empirical data for the candidate probe sequence obtained in the empirical evaluation step described above. In many embodiments, the empirical data employed in the generation of the expression vector are the log ratio values from the sample-pair experiments, as indicated above. Where present, replicate log ratio values may be combined using error-weighted averaging. The combined log ratio data for candidate probes designed to target a single gene are used to populate an expression matrix I, where Iᵢⱼ is the measured expression level of probe i in experiment (condition) j. The number of columns in the expression matrix is the number of experiments performed for empirical validation, the number of rows in the expression matrix is the number of candidate probes designed to target a single gene. The significance of the similarity measure used depends on the number of experimental conditions performed. When Pearson correlation is used to measure the similarity of probes, the expression matrix should consist of at least 4 experiments, at least 8 experiments, at least 12 experiments. The matrix contains only data that survive the processing steps described above. As indicated above, certain feature data may be excluded, leading to missing values in the expression matrix, typically indicated by entering a special value (one that could never arise from an experiment, for example a log ratio of 10\hat{ }6) into the matrix. Subsequent processing steps must be able to process such a matrix.

In the next substep, a similarity matrix is derived or calculated from the obtained expression matrix of the first substep. In this similarity matrix, the entry Sᵢⱼ represents the similarity of the expression vectors for probes i and j. The similarity measure used for this step is independent of the clustering mechanism. Specific examples are Pearson's correlation coefficient (as described in Duda, R. O., and Hart, P. E. (1973). Pattern Classification and Scene Analysis. New York, John Wiley and Sons.), Kendall's rank correlation (as described in Kendall, M. G. (1970). Rank Correlation Methods (4^{th} edition). Griffin and Co. Ltd.), similarity measure based on the Euclidian distance, and weighted Pearson 's correlation.

In the third substep, the candidate probes are clustered into one or more groups based on their similarity indices or matrices, as determined in the previous substep. In other words, the candidate probe sequences are placed into groups based on similar expression patterns. In this substep, a clustering algorithm is typically employed. Several clustering approaches can be applied here, where certain embodiments use the following approach. The input to the algorithm is a pair (S,t) where S is a n-by-n similarity matrix (n is equal to the number of candidate probes and ranges from about 3 to about 20, usually from about 5 to about 12) and t is a user-specified affinity threshold that determines what affinity level is considered significant (where t often ranges from about 0.3 to about 0.9, such as from about 0.5 to about 0.8). The algorithm constructs clusters incrementally and uses average inter-cluster similarity (affinity) between unassigned vertices and the current cluster to make its next decision to add or remove elements from groups. The clusters are “stable” when the average similarity exceeds the affinity threshold (t). In many embodiments, the algorithm allows input of up to 5 t values and iteratively performs the cluster analysis at decreasing affinity thresholds until a cluster of a user-defined minimum size is formed. Cluster members are assigned for each cluster and a cluster size and a cluster quality score is calculated. The quality score of a cluster is a measure of the likelihood of such a cluster occurring if data from unrelated probes from the data set were clustered. Highly unlikely clusters (i.e., those where the data cluster much more tightly than would be expected from data randomly selected according to the distribution of similarity between all probes in the data) are given high scores.

The above clustering protocol and substeps thereof, including the specific representative clustering protocol above that includes affinity value and scoring features) may be performed using any convenient algorithm. Of interest are algorithms that automate the steps of data filtering, data combination, clustering, cluster filtering, and probe selection, e.g., by performing all of the above described substeps. Of particular interest are algorithms that form a non-hierarchical clustering (i.e., the clusters are unrelated and cluster boundaries are determined by the algorithm) and do not assume a given number of clusters (i.e., the number of clusters is determined by the algorithm instead of being a constant given as an input parameter). In certain embodiments, the algorithm employed in this step is a CAST (Cluster Affinity Search Technique) clustering algorithm, as known to those of skill in the art and described in U.S. Pat. No. 6,421,668, the disclosure of which is herein incorporated by reference. See also U.S. Pat. No. 6,468,476, the disclosure of which is herein incorporated by reference, which further discloses Clustering programs or algorithms that may find use in the subject methods.

The above substep results in clustering or grouping of the different candidate probe sequences into two or more groups or clusters of sequences, where each cluster is made up of probe sequences that hybridize to a single target and behave similarly in gene expression experiments, both within a single experimental sample pair and across multiple sample experimental pairs.

In the next step of the illustrative method, a cluster or group produced in the preceding step is identified or selected, i.e., chosen, as the “best” group or cluster, based on at least one criterion, such as affinity threshold, cluster size, cluster score, or combination thereof.

In many embodiments, the protocol employed in this step identifies the “best cluster” based on the affinity threshold used and the size or score of the cluster formed. There are two underlying assumptions in this selection: 1) the expression patterns for the candidate probes are representative of the expression patterns for all the possible probes for a given target; and 2) candidate probes that show gene expression pattern(s) that differ from the pattern shown by the majority of the candidate probes are “outliers”. Thus, for those targets where the candidate probe sequences partition into multiple groups, the goal of the selection strategy of this step is to identify clusters that include a majority of the candidate probes tested. Two representative selection schemes that achieve this goal are described in the examples below. In the first scheme, the “representative (best) cluster” is chosen as the cluster formed at the highest t value that allowed formation of a cluster with at least 5 elements, where elements means candidate probes. These criteria are chosen so that “representative clusters” include at least 60% of the probe sequences tested for a given target sequence, and is twice as large as the second largest cluster. In the second scheme, the “representative cluster” is made up of at least 50% of the candidate probe sequences.

In the final step of the illustrative method, a probe sequence that can be employed in a probe suitable for use as a surface immobilized probe for the target of interest is selected from the identified “best” cluster in the previous step. In other words, a “best” probe sequence is selected from previously identified “best” cluster of candidate probe sequences. (For those targets where a representative cluster is not identified, an optimal probe may be chosen using other criteria, e.g., using the computationally predicted performance of the probes.

In many embodiments, the protocol employed in this step identifies a probe sequence from the representative cluster based on empirical data that demonstrate that the probe is meeting a minimum performance metric. Such metrics may include signal intensities (or processed signal intensities), the confidence measures of the gene expression values, or some combination thereof. Two representative selection schemes are described in greater detail below.

In certain embodiments, a series of filters are applied sequentially. For example, the formation of clusters and selection of a best cluster may be viewed as the first filter. The next filter may be, ranked p-value. Then, the final filter may be based on signal. At each step, probes may be removed that to not meet minimum values, but those that do are not distinguished from each other. In the filtering by P-value step, for example, the binning will specify that any probe with certain performance characteristics is acceptable, and not rank those that are found acceptable. Only on the very last selection step, in this case signal level, is a selection of one probe made.

In the first selection scheme, the probe sequence is selected from the previously identified representative cluster using the confidence measures of the gene expression values for each probe across the experimental set, as computed by an appropriate algorithm, such as the Agilent Feature Extraction program (available from Agilent Technologies, Palo Alto, Calif.), and the maximum signal intensity value obtained for each probe sequence. The initial selection criteria selects candidate probes showing the largest number of gene expression values of highest confidence across the experimental set. P-values are calculated for each combined log ratio value as the probability that the probe shows no differential expression. The P-values are binned by a user-defined P-value threshold so that all P-values within the threshold are equivalent. For each tissue/cell line combination, the candidate probes for each target are ranked by the binned P-values. For each candidate probe, the sum of the ranks is calculated and the candidate probes showing equivalent “sum of ranked P-values” become candidate “OptProbes” that pass onto the next selection criteria. The OptProbe (i.e., the “best” probe) is selected from the candidate OptProbes based on the signal intensity; the maximum mean signal intensity is calculated for each probe across the experimental set and the optimal probe is selected from the candidates as the probe showing the highest maximum mean signal intensity.

In the second scheme, the OptProbe is selected from the representative cluster by choosing the probe showing the minimum (or average or median) P-value across the experimental set.

The above described methodology results in the selection of probe sequences for use in surface immobilized probes that show minimal, if any cross-hybridization.

It should be understood that the above description is intended to illustrate and not limit the scope of the invention. Other aspects, advantages and modifications within the scope of the invention will be apparent to those skilled in the art to which the invention pertains. The following examples are put forth so as to provide those of ordinary skill in the art with examples of how to make and use the method and products of the invention, and are not intended to limit the scope of what the inventors regard as their invention.

### EXAMPLES

The invention is demonstrated further by the following illustrative examples. Parts and percentages are by weight unless otherwise indicated. Temperatures are in degrees Centigrade (° C.) unless otherwise specified. The following preparations and examples illustrate the invention but are not intended to limit its scope. All reagents used herein were from Agilent Technologies, Inc. (Palo Alto, Calif.) or Ambion (Austin, Tex.) or Perkin Elmer Life Sciences (Boston, Mass.) unless indicated otherwise.

Method

All of the experiments were performed and the data obtained using a Mouse Test microarray from Agilent Technologies Inc., Palo Alto, Calif. This microarray contains 10 probes of 2150 mouse genes. The genes were obtained from the Incyte ZooSeq database (Palo Alto, Calif.) and were selected based on uniqueness of the hits for that gene and the number of clones for that gene present in the ZooSeq database.

Linear Amplified targets (Ambion mouse RNA samples from Ambion) were used for hybridization experiments using the aforementioned microarrays according to standard hybridization procedures set forth in the documentation accompanying the microarrays. Nineteen (19) different Tissue Pairs and 4 hybridization replicates/Tissue Pair were carried out. Red Sample indicates that the Tissue Pair was labeled with a red dye (using Cyanine-5 CTP from Perkin Elmer Life Sciences) and Green Sample indicates that the Tissue Pair was labeled with a green dye (using Cyanine-3 CTP from Perkin Elmer Life Sciences). The following Tissue Pairs were employed:

| Red Sample | Green Sample | Red Sample | Green Sample |
|---|---|---|---|
| Brain | Embryo | Ovary | Lung |
| Brain | Heart | Ovary | Testicle |
| Brain | Kidney | RefSample | Embryo |
| Brain | Testicle | RefSample | Liver |
| Brain | Thymus | RefSample | Lung |
| Liver | Brain | RefSample | SME |
| Liver | Embryo | RefSample | Thymus |
| Liver | Kidney | Spleen | Heart |
| Liver | Lung | | |
| Liver | SME | | |
| Liver | Spleen | | |

(SME means Swiss Mouse Embryo cell line)

Following the hybridization, each of the microarrays was scanned using the Agilent scanner (Agilent product # G2565BA). Data was extracted using Agilent Feature Extraction software (Version 6.1.1). The results of the 4 replicates of each experiments were combined. Combined P-values were considered to determine which probes showed a LogRatio different from 0. Based on different studies carried on differential expression and self/self experiments, the LogRatio was considered significantly different from 0 when the combined P-value was lower than 10⁻²⁵.

Using Microsoft Access, we assigned a value of 1 to the probes that had LogRatios significantly different from 0 and a value of 0 to the probes that did not. The minimum number of probes that can form a cluster in the probe validation process referred to above as the Illustrative Method is 4 probes. Thus, it was decided that at least 4 probes per gene must have LogRatios different from 0 to consider that gene a suitable candidate for the probe validation process. If a gene has 4 or more probes with LogRatio values significantly different from 0, that gene was assigned a value of 1, otherwise it was assigned a value of 0. Then, a table was created with 1 or 0 values for each gene for all the 19 different tissue pairs conditions.

A program was employed that allowed analysis of all of the possible combinations of 9 tissue pairs out of the total of 19. First, the program calculated the sum of the values for every gene across the 9 tissue pairs. If the gene had not been considered suitable for the probe validation process in any of the tissue pairs, the sum would be 0. On the other hand, if a gene had been considered appropriate for the probe validation process in all tissue pairs, the sum of the values would be 9. So, for every gene, in each 9 tissue pair combination, a final value is determined that was in the range 0 to 9.

Results

| Gene# | TP1 | TP2 | TP3 | TP4 | TP5 | TP6 | TP7 | TP8 | TP9 | Totals |
|---|---|---|---|---|---|---|---|---|---|---|
| Gene 1 | 0 | 0 | 0 | 0 | 0 | 0 | 0 | 0 | 0 | 0 |
| Gene 2 | 1 | 1 | 1 | 1 | 1 | 1 | 1 | 1 | 1 | 9 |
| Gene 3 | 1 | 0 | 0 | 0 | 0 | 1 | 1 | 0 | 1 | 4 |

The program then calculated the number of 1s, 2s, 3s . . . and 9s for each tissue pairs combination (TPComb).

Results

| TPComb | #0 | #1 | #2 | #3 | #4 | #5 | #6 | #7 | #8 | #9 |
|---|---|---|---|---|---|---|---|---|---|---|
| 1, 2, 3, 4, 5, 6,7.8, 9 | 407 | 133 | 102 | 96 | 128 | 173 | 259 | 347 | 314 | 191 |
| 1, 3, 4, 5, 6, 7, 8, 9, 10 | 408 | 158 | 103 | 98 | 125 | 169 | 254 | 317 | 328 | 190 |
| 1, 4, 5, 6, 7, 8, 9, 10, 11 | 604 | 49 | 39 | 74 | 147 | 214 | 312 | 315 | 264 | 132 |

The best tissue pair combination is the combination with the least number of 0's and the highest number of 9's.

To validate the process and select the optimal tissue pair combination, the combinations with the lowest number of 0's were selected. The lowest value was 407 and all the possible combinations with a value for a number of 0's between 407 and 420 were selected. This allowed analysis of tissue pair combinations with a low number of 0's but with the highest number of 9's. Then, the probe validation software is employed to select the three combinations with the lowest number of 0's, the three combinations with a number of 0's between 407 and 420 with the highest number of 9's, and the three combinations with the highest number of 0's (in theory, the worst combination). These tissue pair combinations were then employed to determine the successfulness of the Probe validation software in finding the optimal probe. Based on the above results, the best tissue pair combinations were the combinations with a number of 0's between 407 and 420 and with the highest number of 9's.

The tissue pair combination selected was:
Brain/Testicle
Liver/Embryo
Liver/Lung
Liver/Spleen
Liver/SME
Ovary/Testicle
RefSample/Liver
RefSample/SME
Spleen/Heart

It is to be understood that the invention is not limited to the particular embodiments of the invention described herein, as variations of the particular embodiments may be made and still fall within the scope of the appended claims. It is also to be understood that the terminology employed is for the purpose of describing particular embodiments and is not intended to be limiting. Instead, the scope of the present invention will be established by the appended claims.

In this specification and the appended claims, the singular forms “a,” “an” and “the” include plural reference unless the context clearly dictates otherwise.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described. Methods recited herein may be carried out in any order of the recited events that is logically possible, as well as the recited order of events.

All publications and patent applications cited in this specification are herein incorporated by reference as if each individual publication or patent application were specifically and individually indicated to be incorporated by reference except insofar as they may conflict with those of the present application (in which case the present application prevails).

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims. Furthermore, the foregoing description, for purposes of explanation, used specific nomenclature to provide a thorough understanding of the invention. However, it will be apparent to one skilled in the art that the specific details are not required in order to practice the invention. Thus, the foregoing descriptions of specific embodiments of the present invention are presented for purposes of illustration and description; they are not intended to be exhaustive or to limit the invention to the precise forms disclosed. Many modifications and variations are possible in view of the above teachings. The embodiments were chosen and described in order to explain the principles of the invention and its practical applications and to thereby enable others skilled in the art to utilize the invention.

1. A method for selecting a combination of nucleic acid sample pairs for evaluating the ability of an oligonucleotide probe to measure differential expression of genes, said method comprising:
(a) conducting differential expression experiments using (i) nucleic acid sample pairs and (ii) nucleic acid probes immobilized on a substrate, said probes representing a set of genes where the number of genes in the set is a portion of an expected number of genes in a sample, and
(b) selecting a nucleic acid sample pair combination in relation to the members of said combination having a maximized number of genes from the set of genes that exhibit differential expression and a minimized number of said genes that do not exhibit differential expression.

2. A method according to claim 1 wherein a determination is made for each gene in step (a) whether the gene is differentially expressed and whether probes for each gene cluster together.

3. A method according to claim 2 wherein said determination is made based on one or more parameters from said differential expression experiments.

4. A method according to claim 3 wherein said parameters are selected from the group consisting of LogRatio, LogRatio error and signal intensities.

5. A method according to claim 3 wherein said parameters are (i) the probability of a combined LogRatio value being significantly different from zero for each of said probes representing a gene and (i) the number of probes for a gene that have a probability of the combined LogRatio value being significantly different from zero above a threshold value.

6. A method according to claim 1 wherein said nucleic acid sample pairs are tissue pairs.

7. A method according to claim 1 wherein in step (a) said differential expression experiments are conducted by contacting a nucleic acid sample pair with a substrate having said nucleic acid probes immobilized thereon.

8. A method according to claim 1 wherein in step (a) said differential expression experiments are conducted by contacting each member of a nucleic acid sample pair with a separate substrate having said nucleic acid probes immobilized thereon.

9. A method for selecting a combination of nucleic acid sample pairs for evaluating the ability of an oligonucleotide probe to measure differential expression of genes, said method comprising:
(a) contacting each nucleic acid sample pair from a plurality of nucleic acid sample pairs with a plurality of probes for each of a predetermined number of genes to determine whether said genes exhibit differential expression,
(b) determining for each gene and each of said nucleic acid sample pairs whether said gene exhibits or does not exhibit differential expression based on one or more parameters,
(c) for a gene that exhibits differential expression in step (b) for a nucleic acid sample pair, assigning a “yes” value, and for each gene that does not exhibit differential expression in step (b) for a nucleic acid sample pair, assigning a “no” value,
(d) tabulating data from step (c) for each combination of said nucleic acid sample pairs to be evaluated, and
(e) selecting a combination of nucleic acid sample pairs having a score based on a maximized number of “yes's” and a minimized number of “no's.”

10. A method according to claim 9 wherein said parameters are selected from the group consisting of LogRatio, LogRatio error and signal intensities.

11. A method according to claim 9 wherein said nucleic acid sample pairs are tissue pairs.

12. A computer-readable medium having recorded thereon a program that carries out steps (b)-(e) of a method according to the method of claim 9.

13. A computational analysis system comprising a computer-readable medium according to claim 12.

14. A method for selecting a combination of nucleic acid sample pairs for evaluating the ability of an oligonucleotide probe to measure differential expression of genes, said method comprising:
(a) contacting each nucleic acid sample pair from a plurality of nucleic acid sample pairs with a plurality of probes for each of a predetermined number of genes to determine whether said genes exhibit differential expression,
(b) determining for each gene and each of said nucleic acid sample pairs whether said gene exhibits or does not exhibit differential expression based on one or more parameters,
(c) analyzing the results of said contacting and said determining utilizing an analysis system comprising a computer, said computer comprising:
(i) means for assigning, for each gene and to each of said nucleic acid sample pairs, a value of 1 if said gene exhibits differential expression or assigning a value of 0 if said gene does not exhibit differential expression,
(ii) means for tabulating data from step (i) to determine the total number scores for each combination of said nucleic acid sample pairs to be evaluated, wherein the maximum number of 1's for each nucleic acid sample pair per gene is the number “n” of nucleic acid sample pairs of said combination, and
(iii) means for selecting a combination of nucleic acid sample pairs based on a score representing a maximized number of n's and a minimized number of 0's.

15. A method according to claim 14 wherein said parameters are selected from the group consisting of LogRatio, LogRatio error and signal intensities.

16. A method according to claim 14 wherein said nucleic acid sample pairs are tissue pairs.

17. A computer-based method for selecting a combination of nucleic acid sample pairs for evaluating the ability of an oligonucleotide probe to measure differential expression of genes, said method comprising:
(a) contacting each nucleic acid sample pair from a plurality of nucleic acid sample pairs with a plurality of probes for each of a predetermined number of genes to determine whether said genes exhibit differential expression,
(b) under computer control, determining for each gene and each of said nucleic acid sample pairs whether said gene exhibits or does not exhibit differential expression based on one or more parameters,
(c) under computer control, for a gene that exhibits differential expression in step (b) for a nucleic acid sample pair, assigning a “yes” value, and for each gene that does not exhibit differential expression in step (b) for a nucleic acid sample pair, assigning a “no” value,
(d) under computer control, tabulating data from step (c) for each combination of said nucleic acid sample pairs to be evaluated, and
(f) under computer control, selecting a combination of nucleic acid sample pairs having a score based on a maximized number of “yes's” and a minimized number of “no's.”

18. A method according to claim 17 wherein said parameters are selected from the group consisting of LogRatio, LogRatio error and signal intensities.

19. A method according to claim 17 wherein said nucleic acid sample pairs are tissue pairs.

20. A method of identifying a sequence of a nucleic acid that is suitable for use as a substrate surface immobilized probe for a target nucleic acid, said method comprising evaluating the sequence using nucleic acid sample pairs selected by a method of claim 1.

21. A method of identifying a sequence of a nucleic acid that is suitable for use as a substrate surface immobilized probe for a target nucleic acid, said method comprising:
(a) identifying a plurality of candidate probe sequences for said target nucleic acid based on at least one selection criterion;
(b) empirically evaluating each of said candidate probe sequences under a plurality of different experimental sets to obtain a collection of empirical data values for each of said candidate nucleic acid probe sequences for each of said plurality of different experimental sets wherein said empirical evaluation employs a nucleic acid sample pair selected by a method according to claim 1;
(c) clustering said candidate probe sequences into one or more groups of candidate probe sequences based on each candidate probe sequence's collection of empirical data values, wherein each of said one or more groups exhibits substantially the same performance across said plurality of experimental sets;
(d) selecting one of said one or more groups based on at least one criterion; and
(e) choosing a candidate probe sequence from said selected group to as said sequence of said nucleic acid that is suitable for use as a substrate immobilized probe for said target nucleic acid.

22. A method of producing an array of nucleic acids on the surface of a substrate, said method comprising:
(a) identifying nucleic acid probes by a method according to claim 21 and
(b) synthesizing or depositing said nucleic acid probes identified in step (a) in an array on the surface of a substrate.

23. A method of detecting the presence of a nucleic acid analyte in a sample, said method comprising:
(a) contacting a nucleic acid array produced according to claim 22 with said sample and
(b) detecting the presence of binding complexes on the surface of said array to detect the presence of said analyte in said sample.

24. A method comprising forwarding data representing a result obtained from a method of claim 23.

25. A method according to claim 23 wherein the data is transmitted to a remote location.

26. A method comprising receiving data representing a result obtained from a method of claim 23.

27. A kit for selecting a combination of nucleic acid sample pairs for evaluating the ability of an oligonucleotide probe to measure differential expression of genes, said kit comprising in packaged combination:
(a) an algorithm for use in conducting differential gene expression experiments using (i) nucleic acid sample pairs and (ii) nucleic acid probes immobilized on a substrate where the probes represent a set of genes, the number of genes in the set being a portion of an expected number of genes in a sample, said algorithm being present on a computer readable medium, and
(b) instructions for using the algorithm to select a nucleic acid sample pair combination in relation to the members of said combination having a maximized number of genes from the set of genes that exhibit differential expression and a minimized number of said genes that do not exhibit differential expression.

28. A kit for identifying a sequence of a nucleic acid that is suitable for use as a substrate surface immobilized probe for a target nucleic acid, said kit comprising:
(a) an algorithm that identifies a sequence of a nucleic acid that is suitable for use as a substrate surface immobilized probe for said target nucleic acid according to the method according to claim 21, wherein said algorithm is present on a computer readable medium; and
(b) instructions for using said algorithm to identify said sequence of a nucleic acid that is suitable for use as a substrate surface immobilized probe for said target nucleic acid.
